(19) United States
(12) Patent Application Publication
(10) Pub. No.: US 2007/0264330 A1
(43) Pub. Date: Nov. 15, 2007

(54) PHARMACEUTICAL FORMULATIONS OF PIMAVANSERIN

(76) Inventors: Bo Ragnar-Tolf, Malmo (SE); Nathalie Schlienger, Frederiksberg (DK); Mikkel Boas Thygesen, Fredensborg (DK)

Correspondence Address: KNOBBE MARTENS OLSON & BEAR LLP, 2040 MAIN STREET, FOURTEENTH FLOOR, IRVINE, CA, 92614, US

(21) Appl. No.: 11/749,110

(22) Filed: May 15, 2007

Related U.S. Application Data
(60) Provisional application No. 60/800,864, filed on May 15, 2006.
(60) Provisional application No. 60/854,665, filed on Oct. 26, 2006.

(51) Int. Cl. A61K 9/20

(52) U.S. Cl. 424/464

(57) ABSTRACT

Disclosed herein are stable pharmaceutical formulations of pimavanserin, which is useful for treating conditions associated with serotonin receptors.

### RELATED APPLICATIONS

This application claims the benefit of U.S. Provisional Application Nos. 60/800,864, filed May 15, 2006, and 60/854,665, filed Oct. 26, 2006, both of which are incorporated herein by reference in their entirety.

### BACKGROUND OF THE INVENTION

1. Field of the Invention

The present invention relates to the fields of medicine and chemistry. More particularly, the present invention relates to pimavanserin and its pharmaceutical formulations and uses.

2. Description of the Related Art

Azacyclic carbamides and carboxylic acid amides constitute a new class of compounds effective in inhibiting an activity of monoamine receptors, including the serotonin receptor of the 5-HT2A subclass. Examples of disease conditions for which such compounds can be used include, but are not limited to, neuropsychiatric diseases such as schizophrenia and related idiopathic psychoses, depression, anxiety, sleep disorders, appetite disorders, affective disorders such as major depressions, bipolar disorder, depression with psychotic features and Tourette's Syndrome. Other beneficial treatments may be drug-induced psychoses and side-effects of Parkinson's disease as well as psychoses secondary to neurodegenerative disorders such as Alzheimer's or Huntington's Disease, hypertension, migraine, vasospasm, ischemia and the primary treatment and secondary prevention of various thrombotic conditions including myocardial infarction, thrombotic or ischemic stroke, idiopathic and thrombotic thrombocytopenic purpura and peripheral vascular disease.

One compound exhibiting promising in vitro and in vivo results for the treatment of conditions associated with inverse agonism or antagonism of 5-HT2A receptors is pimavanserin, which is described in more detail in U.S. Application Publication No. 2004-0213816 (incorporated herein by reference in its entirety). There is a need for stable pharmaceutical formulations of this compound.

### SUMMARY OF THE INVENTION

One embodiment disclosed herein includes a pharmaceutical composition having pimavanserin and at least one pharmaceutically acceptable excipient selected from the group consisting of a sugar, a starch, a cellulose preparation, silicon dioxide aerosol, gelatin, calcium phosphate dibasic, sodium lauryl sulfate, magnesium stearate, sodium stearyl fumarate, talc, polyethylene glycol, and polyvinylpyrrolidone, and combinations thereof. In one embodiment, the pharmaceutically acceptable excipient is selected from the group consisting of a sugar, pregelatinized starch, partially pregelatinized starch, microcrystalline cellulose, silicified microcrystalline cellulose, a lactose-cellulose blend, methyl cellulose, silicon dioxide aerosol, gelatin, calcium phosphate dibasic, sodium lauryl sulfate, magnesium stearate, sodium stearyl fumarate, talc, polyethylene glycol, and polyvinylpyrrolidone, and combinations thereof. In one embodiment, the silicified microcrystalline cellulose is PROSOLV® 90 or PROSOLV® 50. In one embodiment, the silicified microcrystalline cellulose is PROSOLV® HD90. In one embodiment, the silicified microcrystalline cellulose comprises microcrystalline cellulose, colloidal silicon dioxide, colloidal anhydrous silica, and light anhydrous silicic acid. In one embodiment, the partially pregelatinized starch is STARCH 1500®. In one embodiment, the lactose-cellulose blend is CELLACTOSE® 80. In one embodiment, the pharmaceutically acceptable excipient is selected from the group consisting of pregelatinized starch, partially pregelatinized starch, silicified microcrystalline cellulose, a lactose-cellulose blend, methyl cellulose, sodium stearyl fumarate, and polyvinylpyrrolidone, and combinations thereof. In one embodiment, the pimavanserin is pimavanserin tartrate. In one embodiment, the pimavanserin tartrate is crystalline Form A. In one embodiment, the pimavanserin tartrate is crystalline Form C.

One embodiment of the compositions described above includes a silicon dioxide aerosol. In one embodiment, at least about 0.1% by weight of the silicon dioxide aerosol is used. In one embodiment, at least about 0.5% by weight of the silicon dioxide aerosol is used. In one embodiment, at least about 1.0% by weight of the silicon dioxide aerosol is used. In one embodiment, the silicon dioxide aerosol has a specific surface area from about 175 to about 225 m²/g. In one embodiment, the silicon dioxide aerosol is AEROSIL® 200. In one embodiment, the composition also includes lactose, microcrystalline cellulose, and magnesium stearate. In one embodiment, the composition includes at least about 50% by weight lactose, at least about 5% by weight microcrystalline cellulose, and at least about 0.5% by weight magnesium stearate. In one embodiment, the composition includes at least about 65% by weight lactose, at least about 10% by weight microcrystalline cellulose, and at least about 1% by weight magnesium stearate.

Another embodiment of the compositions described above includes lactose, magnesium stearate, and silicified microcrystalline cellulose. In one embodiment, the composition includes at least about 50% by weight lactose, at least about 0.5% by weight magnesium stearate, and at least about 5% by weight silicified microcrystalline cellulose. In one embodiment, the composition includes at least about 65% by weight lactose, at least about 1% by weight magnesium stearate, and at least about 10% by weight silicified microcrystalline cellulose.

Another embodiment of the compositions described above includes partially pregelatinized starch, magnesium stearate, and silicified microcrystalline cellulose. In one embodiment, the composition includes at least about 50% by weight silicified microcrystalline cellulose, at least about 0.5% by weight magnesium stearate, and at least about 5% by weight partially pregelatinized starch. In one embodiment, the composition includes at least about 65% by weight silicified microcrystalline cellulose, at least about 0.5% by weight magnesium stearate, and at least about 5% by weight partially pregelatinized starch. In one embodiment, the composition includes at least about 75% by weight silicified microcrystalline cellulose, at least about 1.0% by weight magnesium stearate, and at least about 5% by weight partially pregelatinized starch.

Some embodiments of any of the compositions described above include an additional antipsychotic agent. In one embodiment, the additional antipsychotic agent is selected from the group consisting of chlorpromazine, mesoridazine, prochlorperazine, thioridazine, Fluphenazine, Perpehnazine, Trifluoperazine, haloperidol, pimozide, clozapine, loxapine, olanzapine, quetiapine, resperidone, ziprasidone, lithium carbonate, Aripiprazole, ETRAFON®, Droperidol, Thioridazine, Thiothixene, Promethazine, Metoclopramide, Chlorprothixene, TRIAVIL®, Molindone, Sertindole, Droperidol, Amisulpride, Melperone, Paliperidone, and Tetrabenazine.

Another embodiment disclosed herein includes a method of treating or preventing a condition selected from the group consisting of a neuropsychiatric disorder, a neurodegenerative disorder, and an extrapyramidal disorder, comprising administering to a subject any of the pharmaceutical compositions described above.

Another embodiment disclosed herein includes a method of reducing a side effect of an antipsychotic agent, comprising administering to a subject any of the pharmaceutical compositions described above. In one embodiment, the side effect is selected from the group consisting of stroke, tremors, sedation, gastrointestinal problems, neurological problems, increased risk of death, cerebrovascular events, movement disorder, dystonia, akathisia, parkinsoniam movement disorder, tardive dyskinesia, cognitive disorders, prolactinemia, catalepsy, psychosis, neuroleptic malignant syndrome, heart problems, pulmonary problems, diabetes, liver failure, suicidality, sedation, orthostatic hypotension, choking, dizziness, tachycardia, blood abnormalities (including abnormal triglyceride levels, increased cholesterol levels, dyslipidemia, and hyperglycemia), syncope, seizures, dysphagia, priapism, thrombotic thrombocytopenic purpura, disruption of body temperature regulation, insomnia, agitation, anxiety, somnolence, aggressive reaction, headache, constipation, nausea, dyspepsia, vomiting, abdominal pain, saliva increase, toothache, rhinitis, coughing, sinusitis, pharyngitis, dyspnea, back pain, chest pain, fever, rash, dry skin, seborrhea, increased upper respiratory infection, abnormal vision, arthralgia, hypoaesthesia, manic reaction, concentration impairment, dry mouth, pain, fatigue, acne, pruritus, myalgia, skeletal pain, hypertension, diarrhea, confusion, asthenia, urinary incontinence, sleepiness, increased duration of sleep, accommodation disturbance, palpitations, erectile dysfunction, ejaculatory dysfunction, orgastic dysfunction, lassitude, increased pigmentation, increased appetite, automatism, increased dream activity, diminished sexual desire, nervousness, depression, apathy, catatonic reaction, euphoria, increased libido, amnesia, emotional liability, nightmares, delirium, yawning, dysarthria, vertigo, stupor, paraesthesia, aphasia, hypoesthesia, tongue paralysis, leg cramps, torticollis, hypotonia, coma, migrain, hyperreflexia, choreoathetosis, anorexia, flatulence, stomatitis, melena, hemorrhoids, gastritis, fecal incontinence, erutation, gastroeophageal reflux, gastroenteritis, esophagitis, tongue discoloration, choleithiasis, tongue edema, diverticulitis, gingivitis, discolored feces, gastrointestinal hemorrhage, hematemesis, edema, rigors, malaise, pallor, enlarged abdomen, ascites, sarcoidosis, flushing, hyperventilation, bronchospasm, pneumonia, tridor, asthma, increased sputum, aspiration, photosensitivity, increased sweating, acne, descreased sweating, alopecia, hyperkeratosis, skin exfoliation, bullous eruption, skin ulceration, aggravated psoriasis, furunculosis, verruca, dermatitis lichenoid, hypertrichosis, genital pruritus, urticaria, ventricular tachycardia, angina pectoris, premature atrial contractions, T wave inversion, ventricular extrasystoles, ST depression, AV block, myocarditis, abnormal accommodation, xerophthalmia, diplopia, eye pain, blepharitis, photopsia, photophobia, abnormal lacrimation, hyponatremia, creatine phosphokinase increase, thirst, weight decrease, decreased serum iron, cachexia, dehydration, hypokalemia, hypoproteinemia, hyperphosphatemia, hypertrigylceridemia, hyperuricemia, hypoglycemia, polyuria, polydipsia, hemturia, dysuria, urinary retention, cystitis, renal insufficiency, arthrosis, synostosis, bursitis, arthritis, menorrhagia, dry vagina, nonpeurperal lactation, amenorrhea, female breast pain, leukorrhea, mastitis, dysmenorrhea, female perineal pain, intermenstrual bleeding, vaginal hemorrhage, increased SGOT, increased SGPT, cholestatic hepatitis, cholecystitis, choleithiasis, hepatitis, hepatocellular damage, epistaxis, superficial phlebitis, thromboplebitis, thrombocytopenia, tinnitus, hyperacusis, decreased hearing, anemia, hypochromic anemia, normocytic anemia, granulocytopenia, leukocytosis, lymphadenopathy, leucopenia, Pelger-Huet anomaly, gynecomastia, male breast pain, antiduretic hormone disorder, bitter taste, micturition disturbances, oculogyric crisis, abnormal gait, involuntary muscle contraction, and increased injury.

Another embodiment disclosed herein includes a pharmaceutical composition comprising pimavanserin tartrate and at least about 0.5% of a lubricant. One embodiment includes at least about 0.8% by weight of the lubricant. One embodiment includes at least about 1% by weight of the lubricant. One embodiment includes at least about 1.5% by weight of the lubricant. One embodiment includes at least about 2% by weight of the lubricant. In one embodiment, the lubricant is magnesium stearate. In one embodiment, the lubricant is sodium stearyl fumarate.

Another embodiment disclosed herein includes a wet granulation formulation for use in preparing tablets, the formulation including pimavanserin tartrate and a non-aqueous granulation solvent. In one embodiment, the non-aqueous granulation solvent comprises ethanol. One embodiment further includes mannitol or lactose, pregelatinized or partially pregelatinized starch, and povidone. One embodiment further includes at least about 65% by dry weight mannitol, at least about 2% by dry weight pregelatinized or partially pregelatinized starch, and at least about 0.5% by dry weight povidone. One embodiment further includes at least about 70% by dry weight mannitol, at least about 5% by dry weight pregelatinized or partially pregelatinized starch, and at least about 1% by dry weight povidone.

Another embodiment disclosed herein includes a wet granulation formulation for use in preparing tablets, the formulation including pimavanserin tartrate and less then about 30% by weight of water. One embodiment includes substantially no water.

Another embodiment disclosed herein includes a method of preparing a pharmaceutical tablet including granulating pimavanserin tartrate using a non-aqueous granulation solvent, drying the granulation, blending the granulation with a lubricant, and compressing the blend into a tablet. In one embodiment, the non-aqueous granulation solvent comprises ethanol. In one embodiment, povidone is dissolved in the non-aqueous granulation solvent. In one embodiment, the granulation further comprises mannitol or lactose, pregelatinized starch, and povidone. In one embodiment, the lubricant comprises magnesium stearate.

Another embodiment disclosed herein includes a method of preparing a pharmaceutical tablet including granulating pimavanserin tartrate using less than about 30% by weight of water, drying the granulation, blending the granulation with a lubricant, and compressing the blend into a tablet.

Another embodiment disclosed herein includes a method of preparing a pharmaceutical tablet including dry blending pimavanserin tartrate with at least one pharmaceutically acceptable excipient selected from the group consisting of a sugar, microcrystalline cellulose, lactose-cellulose blend, calcium phosphate dibasic, silicified microcrystalline cellulose, pregelatinized starch, partially pregelatinized starch, polyvinylpyrrolidone, HPMC, sodium lauryl sulfate, sodium stearyl fumerate, silicon dioxide aerosol, magnesium stearate, talc, polyethylene glycol, and combinations thereof and compressing the blend to form a tablet. One embodiment includes coating the tablet with a taste-masking film.

Another embodiment of the compositions described above includes a pharmaceutical composition having pimavanserin and a pharmaceutically acceptable excipient, wherein the pharmaceutical composition comprises substantially no sodium starch glycolate or sodium croscarmellose. In one embodiment, the pimavanserin is pimavanserin tartrate.

Another embodiment disclosed herein includes a pharmaceutical tablet having a core comprising pimavanserin and a taste-masking film coating over the core. In one embodiment, the film coating is an OPADRY® film. In one embodiment, the pimavanserin is pimavanserin tartrate.

Another embodiment disclosed herein includes a pharmaceutical composition having pimavanserin or a pharmaceutically acceptable salt thereof and a pharmaceutically acceptable excipient, wherein the pharmaceutical composition comprises less than about 0.1% of a compound having the structure of Impurity 2:

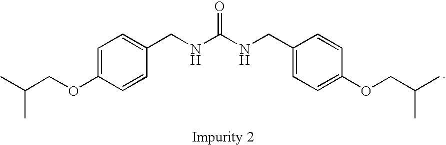

In one embodiment, the pharmaceutical composition is substantially free of Impurity 2. In one embodiment, the pharmaceutical composition is substantially free of Impurity 2 after storage in a blister package at about 30° C. and about 65% relative humidity for at least about 10 weeks.

Another embodiment disclosed herein includes a pharmaceutical composition having pimavanserin or a pharmaceutically acceptable salt thereof and a pharmaceutically acceptable excipient, wherein the pharmaceutical composition comprises less than about 0.25% of a compound having the structure of Impurity 1: 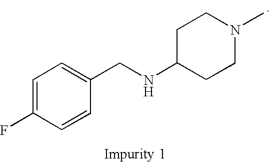
In one embodiment, the pharmaceutical composition comprises less than about 0.25% of Impurity 1 after storage in a blister package at about 40° C. and about 75% relative humidity for at least about 1 month.

Another embodiment disclosed herein includes a pharmaceutical composition, comprising pimavanserin and at least one pharmaceutically acceptable excipient, wherein the composition is formulated such that at least about 80% of the pimavanserin is released from the composition upon administration to a subject. In one embodiment, the composition is formulated such that at least about 90% of the pimavanserin is released from the composition upon administration to a subject.

### BRIEF DESCRIPTION OF THE DRAWINGS

FIG. 1 is a X-ray powder diffraction pattern of crystal Form A of pimavanserin tartrate.

FIG. 2 is a X-ray powder diffraction pattern of crystal Form C of pimavanserin tartrate.

### DETAILED DESCRIPTION OF CERTAIN EMBODIMENTS

It has been surprisingly discovered that certain pharmaceutically acceptable carriers, diluents, and/or excipients when admixed with pimavanserin tartrate result in stable compositions having a long shelf life while other carriers, diluents, and/or excipients result in decreased stability. Accordingly, some embodiments described herein include stable pharmaceutical formulations of pimavanserin tartrate.

Definitions

The term “antagonist” is defined as a compound that competes with an agonist or inverse agonist for binding to a receptor, thereby blocking the action of an agonist or inverse agonist on the receptor. However, an antagonist (also known as a “neutral” antagonist) has no effect on constitutive receptor activity.

The term “inverse agonist” is defined as a compound that decreases the basal activity of a receptor (i.e., signaling mediated by the receptor). Such compounds are also known as negative antagonists. An inverse agonist is a ligand for a receptor that causes the receptor to adopt an inactive state relative to a basal state occurring in the absence of any ligand. Thus, while an antagonist can inhibit the activity of an agonist, an inverse agonist is a ligand that can alter the conformation of the receptor in the absence of an agonist. The concept of an inverse agonist has been explored by Bond et al. in Nature 374:272 (1995). More specifically, Bond et al. have proposed that unliganded β₂-adrenoceptor exists in an equilibrium between an inactive conformation and a spontaneously active conformation. Agonists are proposed to stabilize the receptor in an active conformation. Conversely, inverse agonists are believed to stabilize an inactive receptor conformation. Thus, while an antagonist manifests its activity by virtue of inhibiting an agonist, an inverse agonist can additionally manifest its activity in the absence of an agonist by inhibiting the spontaneous conversion of an unliganded receptor to an active conformation.

The term “5-HT2A receptor” is defined as a receptor, having an activity corresponding to the activity of the human serotonin receptor subtype, which was characterized through molecular cloning and pharmacology as detailed in Saltzman et al., Biochem. Biophys. Res. Comm. 181:1469-78; and Julius et al., Proc. Natl. Acad. Sci. USA 87:928-932.

The term “subject” refers to an animal, preferably a mammal, most preferably a human, who is the object of treatment, observation or experiment.

As used herein, the term “coadministration” of pharmacologically active compounds refers to the delivery of two or more separate chemical entities, whether in vitro or in vivo. Coadministration refers to the simultaneous delivery of separate agents; to the simultaneous delivery of a mixture of agents; as well as to the delivery of one agent followed by delivery of a second agent or additional agents. In all cases, agents that are coadministered are intended to work in conjunction with each other.

Compounds

Pimavanserin, which is also known as N-(1-methylpiperidin-4-yl)-N-(4-fluorophenylmethyl)-N′-(4-(2-methylpropyloxy)phenylmethyl)carbamide, N-[(4-fluorophenyl)methyl]-N-(1-methyl-4-piperidinyl)-N′-[[4-(2-methylpropoxy)phenyl]methyl]-urea, 1-(4-fluorobenzyl)-1-(1-methylpiperidin-4-yl)-3-[4-(2-methylpropoxy)benzyl]urea, or ACP-103 has the structure of formula (I):

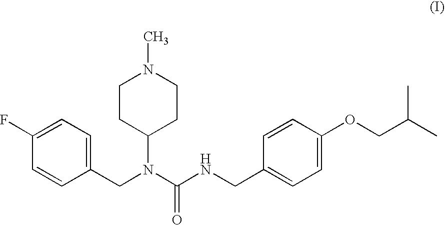

The compound of formula (I) exhibits activity at monoamine receptors, specifically serotonin receptors, and acts as an inverse agonist at the 5-HT2A receptor. Experiments performed on cells transiently expressing the human phenotype of this receptor have shown that the compound attenuates the signaling of such receptors in the absence of additional ligands acting upon the receptor. The compound has thus been found to possess intrinsic activity at this receptor and is able to attenuate the basal, non-agonist-stimulated, constitutive signaling responses that the 5-HT2A receptor displays. The observation that pimavanserin is an inverse agonists also indicates that it has the ability to antagonize the activation of 5-HT2A receptors that is mediated by endogenous agonists or exogenous synthetic agonist ligands. In vivo human and non-human animal studies have further shown that pimavanserin exhibits anti-psychotic, anti-dyskinesia, and anti-insomnia activity.

Unless otherwise indicated, pimavanserin as used herein includes the free base of the compound and all of its salts, hydrates, and polymorphs, either individually or in combination.

Methods of Preparation

The compound of formula (I) may be synthesized by any suitable method, such as is described in U.S. Application Publication Nos. 2004-0213816 and 2006-0106063, both of which are incorporated herein by reference in their entirety. In one embodiment, the compound is synthesized according to the process of Scheme I: 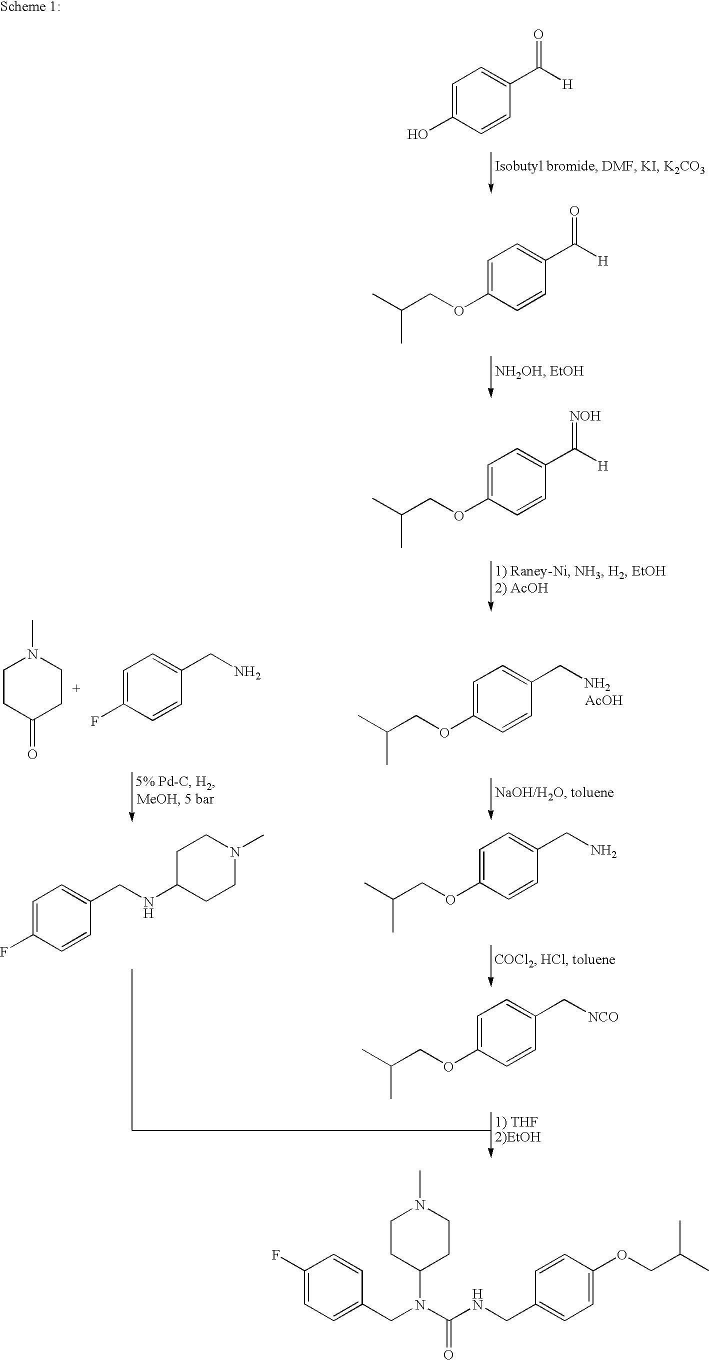
Formation of Pimavanserin Tartrate

The tartrate salt of the compound of formula I has improved solubility in water and hence enhanced bioavailability and improved processing characteristics for the preparation and formulation of drug compositions. The tartrate salt of pimavanserin may be prepared as an integrated part of the process for synthesizing the compound as described above by using tartaric acid as the salt forming acid. Alternatively, the tartrate salt may be formed by reaction of the isolated compound of formula I with tartaric acid.

Crystalline Forms of Pimavanserin (Forms A and C)

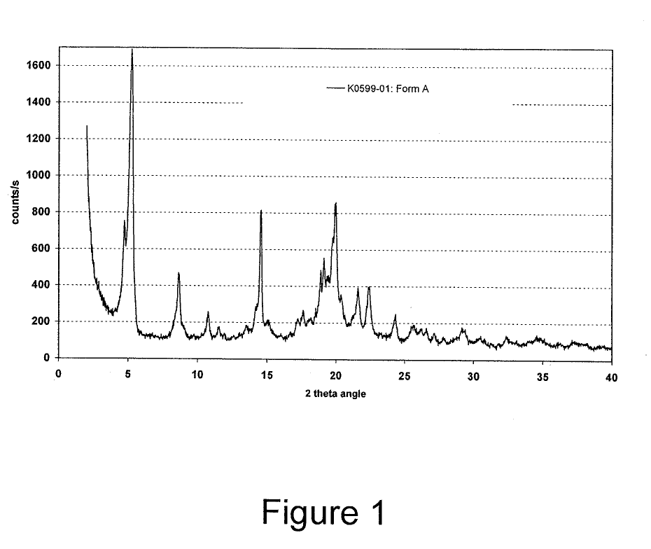

The pimavanserin can be obtained in a number of crystalline forms. One such crystalline form is referred to as crystalline Form A and is described in U.S. Patent Publication No. 2006-0106063, which is incorporated herein by reference in its entirety. The X-ray powder diffraction pattern of Form A is depicted in FIG. 1. Specifically, the X-ray powder diffraction pattern exhibits the following characteristic peaks expressed in d-values (Å): 18.6 (s), 16.7 (vs), 10.2 (s), 8.2 (m), 7.7 (w), 7.4 (w), 6.5 (w), 6.2 (m), 6.1 (vs), 5.86 (w), 5.14 (m), 5.03 (m), 4.78 (m), 4.69 (m), 4.63 (s), 4.49 (s), 4.44 (vs), 4.35 (m), 4.10 (m), 3.96 (s), and 3.66 (m). In various embodiments, Form A is present in a solid form of pimavanserin in amounts of at least about 50%, 70%, 80%, 90%, 95%, or 98%, with the remainder being other crystalline forms (including hydrates and solvates) and/or amorphous forms.

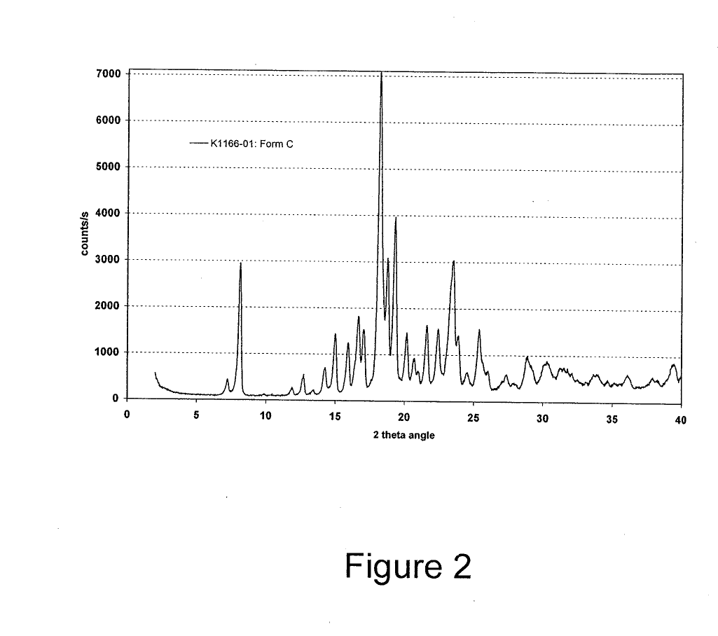

Another crystalline form of pimavanserin is referred to as crystalline Form C and is described in U.S. Patent Publication No. 2006-0106063, which is incorporated herein by reference in its entirety. The X-ray powder diffraction pattern of Form C is depicted in FIG. 2. Specifically the X-ray powder diffraction pattern exhibits the following characteristic peaks expressed in d-values (Å): 12.0 (w), 10.7 (vs), 7.4 (vw), 6.9 (vw), 6.6 (vw), 6.2 (w), 5.86 (m), 5.53 (w), 5.28 (m), 5.16 (m), 4.84 (vs), 4.70 (m), 4.57 (s), 4.38 (m), 4.09 (w), 3.94 (w), 3.77 (s), 3.71 (m), 3.49 (w), 3.46 (w), 3.25 (w), 3.08 (w), and 2.93 (w). In various embodiments, Form C is present in a solid form of pimavanserin in amounts of at least about 50%, 70%, 80%, 90%, 95%, or 98%, with the remainder being other crystalline forms (including hydrates and solvates) and/or amorphous forms.

Some embodiments include a mixture of crystalline forms, which may then be used in the formulations described herein. For example, some embodiments include a mixture of crystalline Form A and crystalline Form C. In some cases, the mixture additionally includes amounts of other crystalline forms (including hydrates and solvates) and/or amorphous forms.

Crystalline Form A can be prepared in a controlled manner by crystallization from ethanol, optionally admixed with isopropanol. Crystalline Form C can be obtained from crystalline Form A by suspension in a polar and aprotic solvent and adding seed crystals of Form C. Crystalline Form A and Form C may be obtained according to Scheme 2; however, any suitable method for obtaining these forms may be used for the formulations described herein. Additional details regarding Form A and Form C and their production may be found in U.S. Application Publication No. 2006-0106063, which is incorporated herein by reference in its entirety.

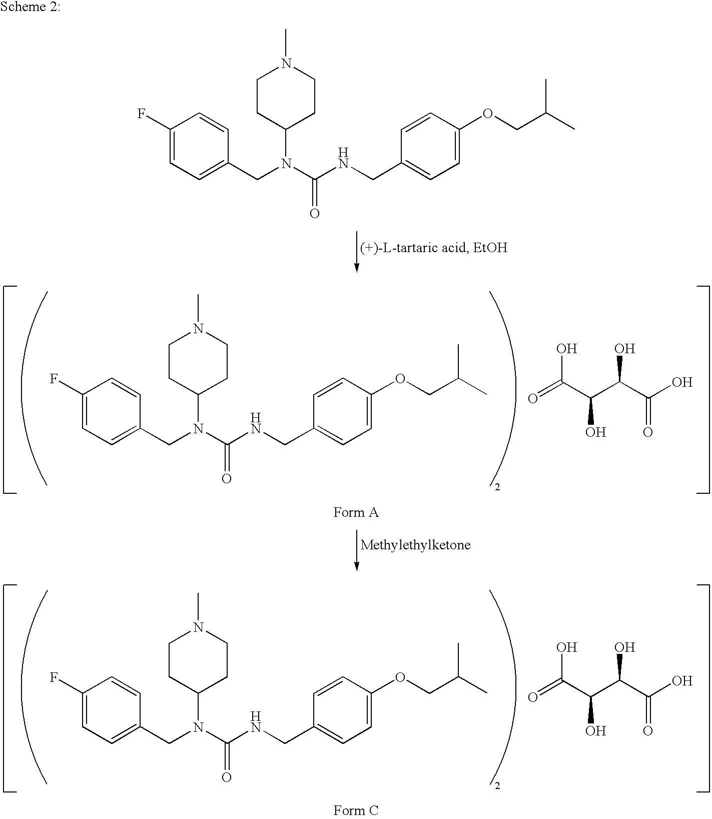

Unless otherwise indicated, as used herein pimavanserin tartrate refers to all its polymorphic and amorphous forms, including crystalline Form A and crystalline Form C, either individually or in combination.

Pharmaceutical Formulations

Some embodiments include a pharmaceutical composition comprising pimavanserin tartrate as described above, and a physiologically acceptable carrier, diluent, or excipient, or a combination thereof.

The term “pharmaceutical composition” refers to a mixture of the compound disclosed herein with other chemical components, such as diluents, carriers, and/or excipients.

The term “physiologically acceptable” defines a carrier, diluent, or excipient that does not abrogate the biological activity and properties of the compound.

The pharmaceutical compositions described herein can be administered to a human patient per se, or in pharmaceutical compositions where they are mixed with other active ingredients (e.g., an anti-psychotic agent, particularly one with dopamine antagonist properties), as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990, which is hereby incorporated by reference in its entirety.

Non-limiting anti-psychotics that may be included in the formulations described herein include a phenothiazine, a phenylbutylpiperadine, a debenzapine, a benzisoxidil, and a salt of lithium. In some embodiments, the phenothiazine is selected from the group consisting of chlorpromazine (Thorazine®), mesoridazine (Serentil®), prochlorperazine (Compazine®), thioridazine (Mellaril), Fluphenazine (Prolixin®), Perpehnazine (Trilafon®), and Trifluoperazine (Stelazine®). In some embodiments, the phenylbutylpiperadine is selected from the group consisting of haloperidol (Haldol®) and pimozide (Orap®). In some embodiments, the debenzapine is selected from the group consisting of clozapine (Clozaril®), loxapine (Loxitane®), olanzapine (Zyprexa®), and quetiapine (Seroquel®). In some embodiments, the benzisoxidil is selected from the group consisting of resperidone (Resperidal®) and ziprasidone (Geodon®). In some embodiments, the salt of lithium is lithium carbonate. In some embodiments, the antipsychotic agent is selected from the group consisting of Aripiprazole (Abilify®), Etrafon®, Droperidol (Inapsine®), Thioridazine (Mellaril®), Thiothixene (Navane®), Promethazine (Phenergan®), Metoclopramide (Reglan®), Chlorprothixene (Taractan®), Triavil®, Molindone (Moban®), Sertindole (Serlect®), Droperidol, Amisulpride (Solian®), Melperone, Paliperidone (Invega®), and Tetrabenazine.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. The compositions can be formulated by combining the active compounds with pharmaceutically acceptable carriers to form tablets, pills, dragees, capsules, liquids, gels, syrups, slurries, suspensions and the like, for oral ingestion by a patient to be treated. For example, pharmaceutical preparations for oral use can be obtained by mixing one or more solid excipient with the drug, optionally grinding the resulting mixture, and processing the mixture of granules, after adding suitable auxiliaries, if desired, to obtain tablets or dragee cores.

Dragee cores may be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. In some embodiments, a taste masking film is coated over a core to mask the bitter taste of the compounds disclosed herein. In some embodiments, the taste masking film is an OPADRY® film.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with a filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Pharmaceutical compositions suitable for use as described herein include compositions where the active ingredients are contained in an amount effective to achieve its intended purpose. More specifically, a “therapeutically effective amount” means an amount of compound effective to prevent, alleviate or ameliorate symptoms of disease or prolong the survival of the subject being treated.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

It has been surprisingly discovered that only certain excipients are compatible with pimavanserin tartrate. Accordingly, some embodiments include formulations that include compatible excipients such as sugars, including lactose (e.g., anhydrous or monohydrate), sucrose, mannitol, or sorbitol; polysaccharides such as, for example, starch (e.g., maize starch, wheat starch, rice starch, or potato starch) including pregelatinized or partially pregelatinized (e.g., STARCH 1500®) starch, and cellulose preparations such as, for example, microcrystalline cellulose (e.g., AVICEL® PH102 or AVICEL® PH112), silicified microcrystalline cellulose (e.g., PROSOLV®, a silicified microcrystalline cellulose blend comprising microcrystalline cellulose, colloidal silicon dioxide, colloidal anhydrous silica, and light anhydrous silicic acid), lactose-cellulose blend (e.g., CELLACTOSE® 80), methyl cellulose, hydroxypropylmethyl-cellulose (HPMC), and sodium carboxymethylcellulose; gelatin; calcium phosphate dibasic; sodium lauryl sulfate; magnesium stearate; sodium stearyl fumarate; talc; polyethylene glycol; and/or polyvinylpyrrolidone (PVP). In some embodiments, the pimavanserin tartrate used in such a formulation is crystalline Form A. In other embodiments, the pimavanserin tartrate is crystalline Form C.

In some embodiments, one or more of the above excipients are combined with pimavanserin tartrate in a dry formulation method. In some such embodiments, crystalline Form A is combined with one or more of a sugar (e.g., lactose or mannitol), a starch, HPMC, sodium lauryl sulfate, magnesium stearate, or polyethylene glycol. In other embodiments, crystalline Form C is combined with one or more of a sugar (e.g., lactose or mannitol), a starch, microcrystalline cellulose, a lactose-cellulose blend, calcium phosphate dibasic, silicified microcrystalline cellulose, sodium lauryl sulfate, magnesium stearate, sodium stearyl fumarate, or talc.

One dry formulation includes pimavanserin tartrate (e.g., crystalline Form A or crystalline Form C) in combination with starch (e.g., STARCH 1500®), silicified microcrystalline cellulose (e.g., PROSOLV® HD90), and magnesium stearate. The active compound, starch, and silicified microcrystalline cellulose may be blended and sifted. Additional silicified microcrystalline cellulose may be added alternately with additional sifting. Such serial dilution can improve content uniformity. Magnesium stearate may then be added and the resulting blended powder compressed into tablets.

Some embodiments include formulations comprising at least about 50% by weight silicified microcrystalline cellulose, at least about 0.5% by weight magnesium stearate, and at least about 5% by weight partially pregelatinized starch. Some embodiments include formulations comprising at least about 65% by weight silicified microcrystalline cellulose, at least about 0.5% by weight magnesium stearate, and at least about 5% by weight partially pregelatinized starch. Some embodiments include formulations comprising at least about 75% by weight silicified microcrystalline cellulose, at least about 1.0% by weight magnesium stearate, and at least about 5% by weight partially pregelatinized starch.

Another dry formulation includes pimavanserin tartrate (e.g., crystalline Form A or crystalline Form C) in combination with a sugar (e.g., lactose or mannitol), magnesium stearate, and silicified microcrystalline cellulose (e.g., PROSOLV®90 or PROSOLV®50 or PROSOLV®HD90). Some embodiments include at least about 50% by weight sugar, at least about 0.5% by weight magnesium stearate, and at least about 5% by weight silicified microcrystalline cellulose. Some embodiments include at least about 65% by weight sugar, at least about 1% by weight magnesium stearate, and at least about 10% by weight silicified microcrystalline cellulose.

Another dry formulation includes pimavanserin tartrate (e.g., crystalline Form A or crystalline Form C) in combination with a sugar (e.g., lactose or mannitol), microcrystalline cellulose, and magnesium stearate. Some embodiments include at least about 50% by weight sugar, at least about 5% by weight microcrystalline cellulose, and at least about 0.5% by weight magnesium stearate. Some embodiments include at least about 65% by weight sugar, at least about 10% by weight microcrystalline cellulose, and at least about 1% by weight magnesium stearate.

In some embodiments, a silicon dioxide aerosol is added during dry formulation to increase flowability. In some embodiments, the amount of silicon dioxide aerosol added is at least about 0.1%, 0.5%, or 1% by weight. In some embodiments, the amount of silicon dioxide aerosol is from about 0.1% to about 2% by weight or from about 0.5% to about 1% by weight. In some embodiments, the silicon dioxide aerosol has a specific surface area from about 175 m²/g to about 225 m²/g.

In some embodiments, the amount of any lubricant added to a dry formulation (e.g., magnesium stearate or sodium stearyl fumarate) is at least about 0.8%, 1%, 1.5%, 2%, or 5% in order to prevent seizing during blending.

In some embodiments, a wet formulation method is used to produce tablets comprising one or more excipients selected from a sugar (e.g., lactose or mannitol), povidone, starch, HPMC, talc, and a lubricant (e.g., magnesium stearate). In some embodiments, the final tabletted composition when using a wet formulation method includes at least about 65% by weight mannitol or lactose, at least about 2% by weight pregelatinized starch, and at least about 0.5% by weight povidone. In some embodiments, the composition includes at least about 70% by weight mannitol or lactose, at least about 5% pregelatinized starch, and at least about 1% by weight povidone. In some embodiments, the amount of mannitol or lactose is from about 60% to about 95% by weight. In some embodiments, the amount of starch is from about 2% to about 10% by weight. In some embodiments, the amount of povidone is from about 0.5% to about 2% by weight. In some embodiments, the final composition includes a lubricant such as magnesium stearate. In some embodiments, the amount of lubricant is at least about 0.5%. In some embodiments, the amount of lubricant is from about 0.5% to about 5% by weight or from about 1% to about 2% by weight.

In some embodiments, tablets produced by any of the methods described above are coated such as with a taste masking film (e.g., an OPADRY® film). In some embodiments, instead of compression into tablets, a dry or wet blend, such as those described above, is placed in a gelatin capsule or HPMC capsule.

In some embodiments, certain excipients are avoided to preserve the stability of the drug substance. In some embodiments, the excipients do not include sodium starch glycolate or sodium croscarmellose. In some embodiments, formulation with water is avoided or the amount of any water present is less than about 30%, 20%, 10%, or 5% by weight. Accordingly, in some embodiments, wet formulation techniques use non-aqueous solvents (e.g., ethanol). In some embodiments at least about 10% by weight of a wet formulation includes a non-aqueous solvent such as ethanol.

In some embodiments, pharmaceutical compositions are provided having low amounts of certain undesirable impurities. In one embodiment, the undesirable impurity is Impurity 1 having the following structure: 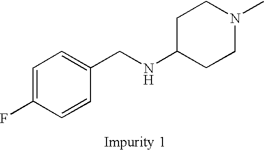
In various embodiments, the amount of Impurity 1 in the pharmaceutical composition is less than about 1%, 0.5%, 0.3%, 0.25%, 0.2%, 0.18%, or 0.17%. In one embodiment, the undesirable impurity is Impurity 2 having the following structure: 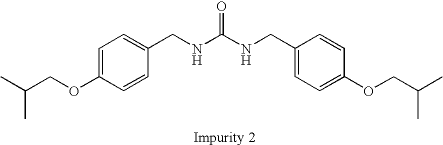
In various embodiments, the amount of Impurity 2 in the pharmaceutical composition is less than about 0.5%, 0.3%, 0.1%, 0.05%, or is low enough that it cannot be detected.

In some embodiments of the any of the above formulations, crystalline Form A of pimavanserin tartrate is used in the formulation. In other embodiments, crystalline Form C is used.

Various embodiments include tablets as described above containing 1 mg, 2 mg, 5 mg, 10 mg, 20 mg, 40 mg, 60 mg, or 80 mg of pimavanserin tartrate.

Some embodiments include tablets as described above formulated for fast release of the pimavanserin. In various embodiments, the tablets are formulated to release at least about 70%, 80%, or 90% of the pimavanserin within 20 minutes of administration of the tablet to a subject.

Methods of Treatment

In some embodiments, a pharmaceutical formulation described above is used to treat or prevent a variety of human conditions. In some embodiments, the condition is a neuropsychiatric disorder, including but not limited to schizophrenia, schizoaffective disorders, mania, depression (including dysthymia, treatment-resistant depression, and depression associated with psychosis), cognitive disorders, aggressiveness (including impulsive aggression), panic attacks, obsessive compulsive disorders, borderline personality disorder, borderline disorder, multiplex developmental disorder (MDD), behavioral disorders (including behavioral disorders associated with age-related dementia), psychosis (including psychosis associated with dementia, induced by treatment, such as treatment of Parkinson's disease, or associated with post traumatic stress disorder), suicidal tendency, bipolar disorder, sleep disorder (including sleep maintenance insomnia, chronic insomnia, transient insomnia, and periodic limb movements during sleep (PLMS)), addiction (including drug or alcohol addiction, opioid addiction, and nicotine addiction), attention deficit hyperactivity disorder (ADHD), post traumatic stress disorder (PTSD), Tourette's syndrome, anxiety (including general anxiety disorder (GAD)), autism, Down's syndrome, learning disorders, psychosomatic disorders, alcohol withdrawal, epilepsy, pain (including chronic pain, neuropathic pain, inflammatory pain, diabetic peripheral neuropathy, fibromyalgia, postherpetic neuralgia, and reflex sympathetic dystrophy), disorders associated with hypoglutamatergia (including schizophrenia, childhood autism, and dementia), and serotonin syndrome.

In some embodiments, the condition is a neurodegenerative disorder, including but not limited to Alzheimer's disease, Parkinson's disease, Huntington's chorea, sphinocerebellar atrophy, frontotemporal dementia, supranuclear palsy, or Lewy body dementia.

In some embodiments, the condition is an extrapyramidal disorder including, but not limited to, dyskinesias (such as induced by treatment of Parkinson's disease), bradykinesia, rigidity, psychomotor slowing, tics, akathisia (such as induced by a neuroleptic or SSRI agent), Friedrich's ataxia, Machado-Joseph's disease, dystonia, tremor, restless legs syndrome, or myoclonus.

In some embodiments, the condition is induced by treatment with an anti-psychotic compound. In other embodiments, the condition is induced by treatment of a neurodegenerative disorder. Accordingly, some embodiments include administering a formulation described herein to alleviate a side-effect associated with treatment of psychosis or a neurodegenerative disorder. For example, in one embodiment, cognitive impairment caused by administration of an antipsychotic is improved by administration of a formulation described herein. In some embodiments, the condition is induced by treatment with a dopamine agonist. Non-limiting examples of conditions induced by dopamine agonists include hiccups, uncontrolled gambling, hypersexual activity, drug cravings, and headache.

In various embodiments, the side effects alleviated by administration of the formulations described herein are selected from the group consisting of stroke, tremors, sedation, gastrointestinal problems, neurological problems, increased risk of death, cerebrovascular events, movement disorder, dystonia, akathisia, parkinsoniam movement disorder, tardive dyskinesia, cognitive disorders, prolactinemia, catalepsy, psychosis, neuroleptic malignant syndrome, heart problems, pulmonary problems, diabetes, liver failure, suicidality, sedation, orthostatic hypotension, choking, dizziness, tachycardia, blood abnormalities (including abnormal triglyceride levels, increased cholesterol levels, dyslipidemia, and hyperglycemia), syncope, seizures, dysphagia, priapism, thrombotic thrombocytopenic purpura, disruption of body temperature regulation, insomnia, agitation, anxiety, somnolence, aggressive reaction, headache, constipation, nausea, dyspepsia, vomiting, abdominal pain, saliva increase, toothache, rhinitis, coughing, sinusitis, pharyngitis, dyspnea, back pain, chest pain, fever, rash, dry skin, seborrhea, increased upper respiratory infection, abnormal vision, arthralgia, hypoaesthesia, manic reaction, concentration impairment, dry mouth, pain, fatigue, acne, pruritus, myalgia, skeletal pain, hypertension, diarrhea, confusion, asthenia, urinary incontinence, sleepiness, increased duration of sleep, accommodation disturbance, palpitations, erectile dysfunction, ejaculatory dysfunction, orgastic dysfunction, lassitude, increased pigmentation, increased appetite, automatism, increased dream activity, diminished sexual desire, nervousness, depression, apathy, catatonic reaction, euphoria, increased libido, amnesia, emotional liability, nightmares, delirium, yawning, dysarthria, vertigo, stupor, paraesthesia, aphasia, hypoesthesia, tongue paralysis, leg cramps, torticollis, hypotonia, coma, migrain, hyperreflexia, choreoathetosis, anorexia, flatulence, stomatitis, melena, hemorrhoids, gastritis, fecal incontinence, erutation, gastroeophageal reflux, gastroenteritis, esophagitis, tongue discoloration, choleithiasis, tongue edema, diverticulitis, gingivitis, discolored feces, gastrointestinal hemorrhage, hematemesis, edema, rigors, malaise, pallor, enlarged abdomen, ascites, sarcoidosis, flushing, hyperventilation, bronchospasm, pneumonia, tridor, asthma, increased sputum, aspiration, photosensitivity, increased sweating, acne, descreased sweating, alopecia, hyperkeratosis, skin exfoliation, bullous eruption, skin ulceration, aggravated psoriasis, furunculosis, verruca, dermatitis lichenoid, hypertrichosis, genital pruritus, urticaria, ventricular tachycardia, angina pectoris, premature atrial contractions, T wave inversion, ventricular extrasystoles, ST depression, AV block, myocarditis, abnormal accommodation, xerophthalmia, diplopia, eye pain, blepharitis, photopsia, photophobia, abnormal lacrimation, hyponatremia, creatine phosphokinase increase, thirst, weight decrease, decreased serum iron, cachexia, dehydration, hypokalemia, hypoproteinemia, hyperphosphatemia, hypertrigylceridemia, hyperuricemia, hypoglycemia, polyuria, polydipsia, hemturia, dysuria, urinary retention, cystitis, renal insufficiency, arthrosis, synostosis, bursitis, arthritis, menorrhagia, dry vagina, nonpeurperal lactation, amenorrhea, female breast pain, leukorrhea, mastitis, dysmenorrhea, female perineal pain, intermenstrual bleeding, vaginal hemorrhage, increased SGOT, increased SGPT, cholestatic hepatitis, cholecystitis, choleithiasis, hepatitis, hepatocellular damage, epistaxis, superficial phlebitis, thromboplebitis, thrombocytopenia, tinnitus, hyperacusis, decreased hearing, anemia, hypochromic anemia, normocytic anemia, granulocytopenia, leukocytosis, lymphadenopathy, leucopenia, Pelger-Huet anomaly, gynecomastia, male breast pain, antiduretic hormone disorder, bitter taste, micturition disturbances, oculogyric crisis, abnormal gait, involuntary muscle contraction, and increased injury. In one embodiment, the side effect is weight gain. In one embodiment, side effect is associated with administration of the antipsychotic to a child under 18. In one embodiment, the side effect in the child is selected from psychosis, schizophrenia, pervasive developmental disorder, autism, Tourette's syndrome, conduct disorder, aggression, attention and hyperactivity difficulties (e.g., ADD, ADHD). In some embodiments, the side effects of weight gain, heart rhythm problems, and diabetes are more severe in children.

Other conditions treatable by the formulations disclosed herein include, but are not limited to, chemotherapy-induced emesis, frailty, on/off phenomena, non-insulin-dependent diabetes mellitus, metabolic syndrome, autoimmune disorders (including lupus and multiple sclerosis), sepsis, increased intraocular pressure, glaucoma, retinal diseases (including age related macular degeneration), Charles Bonnet syndrome, substance abuse, sleep apnea, pancreatis, anorexia, bulimia, disorders associated with alcoholism, cerebral vascular accidents, amyotrophic lateral sclerosis, AIDS related dementia, traumatic brain or spinal injury, tinnitus, menopausal symptoms (such as hot flashes), sexual dysfunction (including female sexual dysfunction, female sexual arousal dysfunction, hypoactive sexual desire disorder, decreased libido, pain, aversion, female orgasmic disorder, and ejaculatory problems), low male fertility, low sperm motility, hair loss or thinning, incontinence, hemorrhoids, migraine, hypertension, thrombosis (including thrombosis associated with myocardial infarction, stroke, idiopathic thrombocytopenic purpura, thrombotic thrombocytopenic purpura, and peripheral vascular disease), abnormal hormonal activity (such as abnormal levels of ACTH, corticosterone, rennin, or prolactin), hormonal disorders (including Cushing's disease, Addison's disease, and hyperprolactinemia), a pituitary tumor (including a prolactinoma), a side effect associated with a pituitary tumor (including hyperprolactinemia, infertility, changes in menstruation, amenorrhea, galactorrhea, loss of libido, vaginal dryness, osteoporosis, impotence, headache, blindness, and double vision), vasospasm, ischemia, cardiac arrythmias, cardiac insufficiency, asthma, emphysema, or appetite disorders.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### EXAMPLES

The invention is disclosed in further detail in the following examples that are not in any way intended to limit the scope of the invention as claimed.

### Example 1

### Preparation of N-(4-fluorobenzyl)-N-(1-methylpiperidin-4-yl)-N′-(4-(2-methylpropyloxy)phenylmethyl)carbamide

a) Preparation of

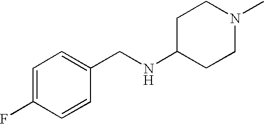

The reaction step was performed in three batches, which were each manufactured on the same scale as described below and the resulting products combined for further use in the next step.

N-Methylpiperidone (33.0 kg) and 4-fluorobenzylamine (35.4 kg) were dissolved in methanol (220.1 kg) at 15-19° C. (exothermic dissolution), and a suspension of 5% palladium on charcoal (1.2 kg) in methanol (16.8 kg) was added under nitrogen and the line rinsed with methanol (5.6 kg). The bulk was heated to 23-27° C. and hydrogenated at the same temperature and ˜5 bar until the hydrogen absorption stopped (˜12 h). The residual starting material was checked by GC, and the bulk was clarified on a Lens filter equipped with a thin Celtrox pad and 2×G92 filter papers. The line was rinsed with methanol (9.8 kg). The solvent was distilled under reduced pressure (265-60 mbar; 35-40° C.) and the oily residue was purified by fractional distillation under vacuum at ˜135-140° C. at 8-0.5 mbar. Impure fractions of the three batches were combined and redistilled.

Total yield (combined three batches and redistilled fractions): 147.4 kg (78.1%).
b) Preparation of

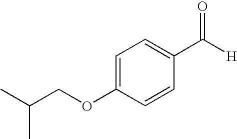

The reaction step was performed in two batches. 4-Hydroxybenzaldehyde (141 kg) was dissolved in dimethylformamide (335 kg) at 15-25° C., then solid potassium carbonate (323 kg) and potassium iodide (19 kg) were added portion wise at <30° C. and the suspension was heated up to 78-82° C. The temperature of the condenser was fixed to −10° C. and isobutylbromide (317 kg) was added to the suspension over 4 h 50 min at 78-82° C. At the end of the addition, the mixture was stirred for 2 h at 78-82° C. and residual starting material was checked by HPLC. The suspension was cooled to 20-30° C., diluted with 100% ethanol (501 kg, denatured with isopropanol), stirred for 15 min at 20-30° C. and centrifuged (3 loadings) to remove the excess of carbonate and potassium bromide. The line and the cake were washed with 100% ethanol (2×32 kg/loading). The solution is used as such in the next step.
c) Preparation of

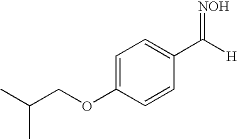

To the aldehyde solution resulting from Step b, 50% hydroxylamine in water (115 kg) was added at room temperature over ˜0.5 h (the addition is slightly exothermic), the line washed with ethanol (8 kg), then the bulk was heated up to 73-77° C. and stirred at this temperature for 2 h. The bulk was concentrated under reduced pressure (250-120 mbar, 45-55° C.) to ˜850 L, the residue quenched with water (951 kg) at 45-55° C. and the residual ethanol distilled under vacuum (270-150 mbar, 45-55° C., residual volume=1466 L). The bulk was diluted with petrol ether 60-90 (557 kg) and heated at reflux (˜60° C.) to reach complete dissolution (˜20 min, visual check). The solution was cooled down to 8-12° C. (crystallization occurs at T=˜27° C.) over ˜5.5 h. After 0.5 h stirring at 10° C., the mixture was cooled to 0-5° C. and stirred at this temperature for 2 h. The bulk was centrifuged (3 loadings) and the cake washed with petrol ether (2×23 kg/loading), then dried under reduced pressure at 40° C. to afford the crude oxime (212 kg).

Recrystallization:

The crude product (212 kg) was dissolved in hexane (642 kg) at 15-25° C. and the suspension heated up to ˜62° C. Charcoal (6 kg) in hexane (26 kg) was added and the suspension was stirred for 0.5 h. After filtration (the filter was washed with 33 kg hexane), the solution was cooled to crystallisation temperature (˜55° C.), and the mixture was stirred for 1 h at this temperature. The suspension was cooled to 10-15° C. After stirring for ˜2 h at that temperature, the bulk was centrifuged (3 loadings) and the cake washed with cold hexane (2×13 kg/loading), then dried under reduced pressure at 40° C.

Yield oxime: 196 kg (87.9% over the two steps)
d) Preparation of

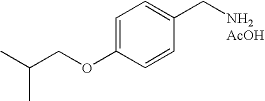

The oxime (198 kg) from Step c was dissolved in ethanol (1148 kg, denatured with isopropanol). Raney nickel catalyst (29 kg) was washed with ethanol (692 kg) until the water content by Karl Fischer was below 300 ppm, then the anhydrous Raney-Nickel was added under nitrogen to the oxime solution, the line washed with ethanol (62 kg) and the suspension cooled down to −10° C. Ammonia gas (229 kg) was added under vacuum over ˜6 h (the addition is exothermic). Then the suspension was heated to 49° C. The internal pressure increased to ˜3 bar. The bulk was hydrogenated at 49° C. and 4 bar until the hydrogen absorption stopped (˜9 h) and the end of reaction was checked by HPLC. The suspension was cooled to 13° C., the excess of ammonia was removed, and the bulk clarified by filtration over Celtrox (4 kg). The line was washed with ethanol (317 kg). The solvent was distilled under reduced pressure (150-10 mbar, 40-50° C.) and the residue dissolved in toluene (780 kg) at ˜40° C. The solution was transferred to a new reactor (previous reactor washed with 57 kg toluene), and cooled to 22° C. Acetic acid (60 kg) was slowly added (exothermic reaction) at 22° C. and the bulk heated during 20 min to ˜95° C. until complete dissolution was reached. The solution was cooled rapidly to 80° C. and seeded with amino acetate product (50 g). The suspension was stirred at the crystallization temperature for 30 min, cooled to 10° C. and stirred for ˜1 h at this temperature. The bulk was centrifuged (3 loadings) and the cake washed with cold toluene (2×48 L/loading) and finally dried under vacuum at (9-16 mbar) at ˜50° C. for 28 h.

Yield: 207 kg (83.6%)
e) Preparation of

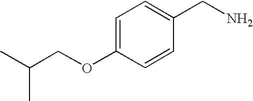

A solution of the aminoacetate (269 kg) from Step d in water (431 kg) was basified with 30% sodium hydroxide solution (305 kg) to pH 14 at 20-25° C. Then the amino base product was extracted with toluene (933 kg) at 43-47° C. by stirring for 15 min. The bulk was decanted during 15 min at 43-47° C.; if necessary the pH was adjusted to >12 with additional 30% NaOH, then the layers were separated. The organic layer was washed with water (359 kg), then concentrated under vacuum (200-20 mbar) at 45-50° C. to give the aminobase as an oily residue.
f) Preparation of

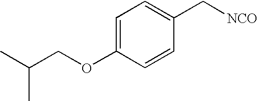

The aminobase from Step e was dissolved at 48° C. in toluene (825 kg) and the water content of the solution checked (KF<300 ppm). The toluene solution was cooled to 1-5° C. and hydrogen chloride (gas, 45.1 kg) was slowly introduced during ˜3 h through a canula at Tₘₐₓ=10° C. (gas introduction is very exothermic). At the end of the addition, the bulk was heated up to 97-103° C. and phosgene (166 kg) was slowly introduced (˜4 h) through a canula. At the end of the addition, the bulk was cooled down to 80-84° C. and the reaction was checked by TLC. Additional phosgene (16 kg) was introduced at 100° C., upon which the bulk turned to a clear solution. After further stirring of the mixture 1 h at 100° C., the bulk was cooled to 80-84° C. The solution was concentrated under vacuum (250-50 mbar) at the same temperature to 770 L. The bulk was checked for the absence of residual phosgene and the crude isocyanate solution in toluene was cooled to 20-25° C., filtered through a cartridge filter 0.3 micron.

Yield: Toluene solution of the isocyanate: 687 kg (34.7% a/a of product by GC), 234.4 kg product (100%, over Steps e and f).
g) Preparation of the Title Compound

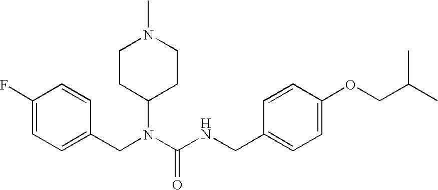

A solution of the isocyanate from Step f in toluene (301 kg, ˜34%) was added in 30 min to a solution of the fluoramine (109 kg) from Step a in tetrahydrofuran (948 kg) at 40° C. and the line washed with tetrahydrofuran (48 kg). The mixture was stirred for ˜3 h until complete dissolution. Residual fluoramine was checked by TLC, and an additional amount of the isocyanate solution (6 kg, ˜34% in toluene) was added and the mixture stirred for 1 h at 40° C. and checked again by TLC. The solvent was removed by distillation under reduced pressure (300-20 mbar) at Tjacket=50° C. Ethanol (663 kg) was added to the residue at 25° C. and the mixture heated to 40-45° C. over 2.5 h and stirred at this temperature for ˜2 h until complete dissolution.

### Example 2

### Preparation of the tartrate salt of N-(4-fluorobenzyl)-N-(1-methylpiperidin-4-yl)-N′-(4-(2-methylpropyloxy)phenylmethyl)carbamide

A previously prepared solution of tartaric acid (41 kg) in ethanol (480 kg) at 43° C. was added at 43° C. over 40 min to the ethanol solution produced in Example 1(g) and the line washed with 16 kg ethanol. The solution was cooled to 37° C. and seeded with pimavanserin Form C (0.5 kg) and the product crystallized at ˜34° C. The suspension was stirred at this temperature for 30 min then cooled to 2° C. over 2.5 h and stirred for 2.5 h more at this temperature. The product was centrifuged (2 loadings) and the cake was washed with ethanol (3×15 kg/loading). The obtained crude product was dried under vacuum (50 to 5 mbar) at 45° C. for about 49 h 20 min, sieved at 3 mm, and dried for another 5 h under vacuum.

Yield of crude: 214 kg (86.0%).

### Example 3

### Preparation of Crystalline Form A of Pimavanserin

The crude tartrate (212 kg) salt from Example 2 was heated in ethanol (948 kg) at 73-75° C. (reflux) for ˜1 h until dissolution. The hot mixture was filtered through a 0.3 μm cartridge filter, the line washed with ethanol (30 kg) and the bulk heated to reflux for ˜0.5 h. The solution was cooled over ˜1 h to 49° C. and seeded with pimavanserin (0.4 kg) and the product crystallized at 48° C. The suspension was stirred at this temperature for 30 min. The suspension was then cooled to −10° C. over ˜8 h and stirred at this temperature for an additional 8 h. The product was centrifuged (2 loadings) and the cake washed with cold ethanol (3×21 kg/loading). The wet product was dried under vacuum (50 to 5 mbar) at 45° C. for 40.5 h. The resulting product was reworked according to the procedure described below. Yield: 189 kg (89.2%)

Rework:

Step #1: Free Basing of the Tartrate to Isolate Urea as a Solid

NaOH 30% (50 kg) was added over ˜15 min to a suspension of water (378 kg), toluene (983 kg) and the tartrate salt (189 kg). The mixture was heated and stirred at 38° C. for 45 min, additional NaOH 30% (6 kg) was added for the pH to reach 12-14. The mixture was stirred at 38° C. for 30 min until complete dissolution and the pH checked. Then, the reaction mixture was settled at 38° C., the layers were separated and the aqueous layer was discarded. The organic layer was washed with water (378 kg) at 38° C. and the toluene distilled at 45-50° C. under vacuum (200-80 mbar) to ˜380 L. Heptane (776 kg) was added to the distillation residue at 48° C. to crystallize the urea. The suspension was stirred at 50° C. for 30 min, then cooled to 1° C. over ˜3 h and stirred at this temperature for 1 h. The product was centrifuged (2 loadings) and the cake washed with cold heptane (2×27 kg/loading). The wet product (urea) was dried under vacuum (40 to 1 mbar) at 50° C. for about 12 h and sieved at 2 mm. Yield: 147 kg (91.5%)

Step #2: Re-Formation of the Tartrate by Addition of Tartaric Acid

The urea (147 kg) in ethanol (535 kg) was stirred at 40-45° C. until complete dissolution, the solution filtered over a 0.3 μm cartridge and the line washed with ethanol (59 kg). A solution of tartaric acid (26.3 kg) in ethanol (223 kg) was added over 40 min through a 0.3 μm cartridge to the solution of the urea (147 kg, from Step #1) in ethanol (594 kg) at 40-45° C., and the line and reactor washed with ethanol (19 kg). The product crystallized during the introduction. The suspension was stirred at 43° C. for 30 min then cooled to −5° C. over ˜6 h and stirred at this temperature for 2 h. The product was centrifuged (3 loadings) and the cake washed with cold ethanol (2×19 kg/loading). The wet product was dried under vacuum (40-7 mbar) at 45° C. for about 34 h, sieved at 3 mm, and drying continued (20-7 mbar, 45° C.) for additional 6 h to produce dry crystalline Form A. Yield: 167 kg (96.8%).

### Example 4

### Preparation of Crystalline Form C of Pimavanserin

A suspension of crystalline Form A (167 kg) from Example 3 in pre-filtered and degassed methylethylketone (942 kg) was heated to 60° C. and stirred at this temperature for ˜2 h. The suspension was seeded with a suspension of crystalline Form C (5.6 kg) in methylethylketone (41 kg, filtered and degassed) and stirred at 60° C. for another 12 h. A sample was taken to check the complete conversion into Form C. The mixture was cooled down to 15° C. over 4.5 h and stirred at this temperature for 2 h; then the product was centrifuged (2 loadings) and the cake washed with cold methylethylketone (2×34 kg/loading). The wet product was dried for 1 h at 45° C., then under vacuum (500 mbar to maximum over 5 h) at 45° C. for ˜18.5 h and the product sieved at 3 mm and packaged. Yield: 160 kg (95.8%).

### Example 5

### Preparation of Crystalline Form A

Pimavanserin tartrate (3.04 kg) was slurried in ethanol (18.2 L). The slurry was heated at 75° C. until it dissolved. The solution was filtered on a cartridge filter and the filter was rinsed with ethanol (0.9 L). The solution was cooled over 1 h to 55° C. and seeded with crystalline Form A of pimavanserin tartrate (0.02 kg). The suspension was cooled to −10° C. in 3 h and stirred at this temperature for 2 h. The product was centrifuged and the cake was washed with cold ethanol (2×1.5 L). The wet cake was dried at 25-30° C. for 5 days to obtain 2.8 kg of product (yield=92.4%).

It was discovered that the yield of manufacturing scale product was improved when the temperature of the suspension was decreased (e.g., to about 10° C. or less, about 0° C. or less, or about −10° C. or less as in this example). Prior methods used a temperature of about 20° C. with lower yields (about 87%).

### Example 6

### Preparation of Crystalline Form C

A suspension of pimavanserin tartrate (8M) in pre-filtered and degassed methyl ethyl ketone was heated to 60° C. and stirred for 8 h under nitrogen atmosphere. The mixture was cooled to 15° C. over 4.5 h and stirred for 2 h, then the product was centrifuged and the cake washed with cold (15° C.) prefiltered and degassed methyl ethyl ketone. The wet product was dried for 15 h in vacuo at 45° C., discharged, packaged under nitrogen and stocked at 0 to 4° C. By using an oxygen free environment, oxidation of product was prevented and complete conversion to polymorph Form C was observed after 2 hours of stirring at 60° C. Yield 95.1%.

### Example 7

### Excipient Compatibility Study with Pimavanserin Tartrate Crystalline Form A

Physical mixtures of crystalline Form A and fourteen processing excipients, gelatin and hydroxypropyl methylcellulose capsules, and drug substance alone were prepared. These excipients tested along with the excipient:drug ratio are listed in Table 1.

TABLE 1
Excipients tested for compatibility with pimavanserin tartrate

| | Excipient:Drug |
|---|---|
| Excipient | Ratio |
| Lactose Anhydrous | 9:1 |
| Microcrystalline Cellulose PH102 | 9:1 |
| Calcium Phosphate Dibasic | 9:1 |
| Microcrystalline Cellulose/Silicon dioxide | 9:1 |
| (PROSOLV ® 90) | |
| Pregelatinized Starch 1500 | 1:1 |
| Polyvinylpyrrolidone (K29/32) | 2:1 |
| Hydroxypropyl Methylcellulose (ESP) | 2:1 |
| Sodium Croscarmellose (AC-DI-SOL ®) | 1:1 |
| Sodium Starch Glycolate (EXCPLOTAB ®) | 1:1 |
| Sodium Lauryl Sulfate | 1:1 |
| Colloidal Silicon Dioxide | 1:1 |
| Magnesium Stearate | 1:1 |
| Talc | 1:1 |
| Polyethylene Glyco13350 | 4:1 |
| Gelatin Capsule, White, Size #0 | 1.8:1 |
| HPMC Capsule, White #8, Size #0 | 1.8:1 |

Binary mixtures of drug with each excipient were prepared by physically mixing 50.0 mg (±2.0 mg) of drug substance with the specified proportion of each excipient. The stability of the drug in the presence of each excipient was studied in dry samples as well as with 30% w/w (relative to bulk drug weight) de-ionized water. Each sample group was stored under the following conditions: 5° C. (for time=0 testing), 40° C./75% RH (relative humidity) one month, and 40° C./75% RH three months.

Bulk drug substance was weighed and added to a sample vial. De-ionized water was then measured (0.015 mL) and added to the vials designated as wet samples. The wet samples were then mixed using a vortex mixer for approximately 15 seconds to incorporate the water and the drug. Excipient was then weighed and added to appropriate vials. The contents of each vial were mixed for approximately 15 seconds using a vortex mixer to produce a uniform mixture of (wet or dry) drug/excipient.

To prepare the capsule samples, the bulk drug substance was weighed directly into the capsule. The capsules were then placed into appropriate vials. Fifteen microliters of de-ionized water was added to the inside of capsules designated as wet samples.

All samples were stored in 20 mL Type I clear glass scintillation vials with foil-lined screw-type vial caps were tightened by hand.

Samples were stored in controlled room temperature/humidity until ready for testing. Other sample vials were stored at 40° C./75% RH unprotected from light and tested at one and three months.

The chromatographic conditions used to evaluate the pimavanserin tartrate content were as follows:

Column: Waters Symmetry C18 (3.5 μm), 150×4.6 mm

Mobile Phase:
Mobile Phase A: Water with 0.1% TFA
Mobile Phase B: Acetonitrile with 0.1% TFA

Flow Rate: 1.0 mL/minute

Injection Volume: 20 μL

Detection: UV @226 nm

Run Time: 35 minutes

Column Temperature: 35° C.

| | Time (min) | % Mobile Phase A | % Mobile Phase B |
|---|---|---|---|
| Gradient | 0 | 85 | 15 |
| Program: | 30 | 0 | 100 |
| | 31 | 0 | 100 |
| | 33 | 85 | 15 |
| | 35 | 85 | 15 |

Sample Diluent: Acetonitrile:Water (1:1)

The contents of each vial were transferred using a funnel into 50 mL volumetric flasks. The vials were rinsed several times with diluent and added to the volumetric flasks. The contents of the capsules were transferred to 50 mL volumetric flasks and the capsule shells were also rinsed with diluent. Diluent was added to the volumetric flasks and they were sonicated for 10 minutes, then diluted to volume with diluent. The solutions were further diluted 5.0 mL into a 50 mL volumetric flask and brought to volume with diluent. These diluted solutions were used for the assay. All solutions were filtered through 0.45 micron PTFE filters, with the first 1-2 mL discarded before filling the HPLC autosampler vials. The diluted sample solutions were assayed against a reference standard of drug product prepared in a similar manner.

The results for the dry samples are listed in Table 2. The results indicated that samples containing the following excipients have potential for interaction: calcium phosphate dibasic, hydroxypropyl methylcellulose (METHOCEL® E5P), sodium croscarmellose, sodium starch glycolate, magnesium stearate, the gelatin capsule, and hydroxypropyl methylcellulose capsule. Some samples yielded low assay values after three months storage at 40° C./75% RH. Sodium croscarmellose and sodium starch glycolate in particular had a significant amount of interaction with the drug.

TABLE 2
Assay results for excipient compatibility study of crystalline Form A
under dry conditions.

| | | % Drug | % Drug |
|---|---|---|---|
| | | One | Three |
| | | Month | Months |
| | % Drug | 40° C./ | 40° C./ |
| Excipient | (time = 0) | 75% RH | 75% RH |
| # 1 Lactose Anhydrous | 96.8 | 92.9 | 95.2 |
| #2 Microcrystalline Cellulose | 99.3 | 95.2 | 94.3* |
| (AVICEL ® PH102) | | | |
| #3 Calcium Phosphate Dibasic | 98.5 | 95.5 | 91.8* |
| #4 MCC-SiO₂ Blend | 99.8 | 95.4 | 92.6* |
| (PROSOLV ® 90) | | | |
| #5 Pregelatinized Starch 1500 | 98.1 | 96.4 | 96.6 |
| #6 Polyvinylpyrrolidone (K29/32) | 99.5 | 96.4 | 95.5* |
| #7 HPMC | 96.5 | 93.7 | 95.6 |
| (METHOCEL ® E5P) | | | |
| #8 Sodium Croscarmellose (AC-DI- | 76.6 | 70.8 | 72.5** |
| SOL ®) | | | |
| # 9 Sodium Starch Glycolate | 88.1 | 90.3 | 88.6** |
| (EXPLOTAB ®) | | | |
| #10 Sodium Lauryl Sulfate | 97.6 | 97.4 | 98.3 |
| #11 Colloidal Silicon Dioxide | 98.9 | 95.4 | 94.9* |
| #12 Magnesium Stearate | 98.8 | 97.4 | 97.2 |
| #13 Talc | 98.9 | 97.3 | 96.6* |
| #14 Polyethylene Glycol 3350 | 98.5 | 97.5 | 98.6 |
| #15 Gelatin Capsule, White, Size #0 | 95.3 | 94.0 | 96.5 |
| #16 HPMC Capsule, | 94.7 | 96.2 | 95.3 |
| White #8, Size #0 | | | |
| #17 Drug Substance | 98.4 | 94.9 | 95.9 |

*These components show a trend in decreasing API assay values.
**These items indicate a strong incompatibility with the drug compound.

Table 3 lists the assay results of drug alone and in the presence of the listed excipients with 30% w/w water added. It was discovered that wet samples are generally less stable than dry materials. In general, if formulating with water, microcrystalline cellulose, calcium phosphate dibasic, sodium croscarmellose, sodium starch glycolate, gelatin capsule, and HPMC capsule may be problematic. The low assay value of drug alone over time indicates that formulating with water in general may be problematic.

TABLE 3
Assay results for excipient compatibility study of crystalline Form A
under wet conditions.

| | | % Drug | % Drug |
|---|---|---|---|
| | | One | Three |
| | | Month | Months |
| | % Drug | 40° C./ | 40° C./ |
| Excipient | (Time = 0) | 75% RH | 75% RH |
| # 1 Lactose Anhydrous | 98.6 | 98.2 | 94.8 |
| #2 Microcrystalline Cellulose | 98.2 | 90.5 | 89.7 |
| (AVICEL ® PH 102) | | | |
| #3 Calcium Phosphate Dibasic | 96.5 | 94.0 | 92.9 |
| #4 MCC-SiO₂ Blend | 95.6 | 94.1 | 94.5 |
| (PROSOLV ® 90) | | | |
| #5 Pregelatinized Starch 1500 | 96.1 | 95.7 | 95.5 |
| #6 Polyvinylpyrrolidone (K29/32) | 96.9 | 93.4 | 94.1 |
| #7 HPMC (METHOCEL ® E5P) | 96.0 | 95.4 | 96.3 |
| #8 Sodium Croscarmellose (AC- | 75.3 | 75.3 | 74.5 |
| DI-SOL ®) | | | |
| # 9 Sodium Starch Glycolate | 88.4 | 89.0 | 88.3 |
| (EXPLOTAB ®) | | | |
| #10 Sodium Lauryl Sulfate | 97.5 | 97.6 | 97.2 |
| #11 Colloidal Silicon Dioxide | 94.5 | 97.3 | 95.7 |
| #12 Magnesium Stearate | 98.4 | 97.9 | 93.2 |
| #13 Talc | 97.0 | 97.7 | 95.4 |
| #14 Polyethylene Glycol 3350 | 97.5 | 99.3 | 97.2 |
| #15 Gelatin Capsule, White, | 56.9 | 93.2 | 95.1 |
| Size #0 | | | |
| #16 HPMC Capsule, White #8, | 44.0 | 96.0 | 95.6 |
| Size #0 | | | |
| #17 Drug Substance | 98.5 | 91.2 | 93.5 |

Baseline HPLC measurements were made on excipients only for comparison to the excipient with drug substance to determine if there was any interference caused by the excipients which could be responsible for inaccurate assay numbers. Chromatograms show virtually no added interference caused by any of the excipients studied as demonstrated by a comparison of the excipient only to time=0 and one month drug/excipient samples.

Total Related Substance (TRS) data was obtained for both the dry and wet studies and are listed in Table 4. For the dry mixtures, the 3-month TRS for the lactose, starch, HPMC, sodium lauryl sulfate, magnesium stearate, polyethylene glycol, gelatin capsule, and HPMC capsule mixtures indicated values approximately less than or equal to that of drug alone, indicating that these materials could be suitable candidates for dry formulations as they tend to show a stabilizing effect on the drug substance. For the wet mixtures, the 3-month TRS for starch, HPMC, talc, gelatin capsule, and HPMC capsule mixtures indicated values approximately less than or equal to that of drug alone indicating their suitability.

TABLE 4
Total related substance data for drug/excipient sample.

| | Time 0 - | 1 month - | 3 month - | Time 0 - | 1 month - | 3 month - |
|---|---|---|---|---|---|---|
| Excipient | Dry | Dry | Dry | Wet | Wet | Wet |
| #1 Lactose Anhydrous | 0.37 | 0.98 | 2.31 | 0.36 | 1.53 | 3.13 |
| #2 Microcrystalline | 0.36 | 1.60 | 3.47 | 0.37 | 2.20 | 3.63 |
| Cellulose (AVICEL ® | | | | | | |
| PH102) | | | | | | |
| #3 Calcium Phosphate | 0.36 | 1.85 | 3.94 | 0.37 | 2.58 | 3.77 |
| Dibasic | | | | | | |
| #4 MCC-SiO₂ Blend | 0.37 | 1.73 | 4.05 | 0.38 | 1.88 | 3.79 |
| (PROSOLV ® 90) | | | | | | |
| #5 Pregelatinized Starch | 0.36 | 1.37 | 2.81 | 0.38 | 2.02 | 3.21 |
| 1500 | | | | | | |
| #6 Polyvinylpyrrolidone | 0.36 | 1.08 | 3.23 | 0.38 | 1.20 | 4.17 |
| (K29/32) | | | | | | |
| #7 HPMC (METHOCEL ® | 0.35 | 1.04 | 1.90 | 0.37 | 1.66 | 2.94 |
| E5P) | | | | | | |
| #8 Sodium Croscarmellose | 0.46 | 2.02 | 5.02 | 0.44 | 2.42 | 5.80 |
| (AC-DI-SOL ®) | | | | | | |
| #9 Sodium Starch | 0.40 | 1.24 | 3.18 | 0.39 | 2.40 | 4.00 |
| Glycolate (EXPLOTAB ®) | | | | | | |
| #10 Sodium Lauryl Sulfate | 0.34 | 0.85 | 1.10 | 0.34 | 1.68 | 3.58 |
| #11 Colloidal Silicon | 0.36 | 1.76 | 3.85 | 0.32 | 2.11 | 3.79 |
| Dioxide | | | | | | |
| #12 Magnesium Stearate | 0.35 | 1.18 | 2.23 | 0.36 | 1.89 | 3.46 |
| #13 Talc | 0.35 | 1.41 | 2.81 | 0.36 | 1.71 | 3.32 |
| #14 Polyethylene Glycol | 0.36 | 0.97 | 1.41 | 0.35 | 0.75 | 1.30 |
| 3350 | | | | | | |
| #15 Gelatin Capsule, | 0.27 | 1.37 | 2.66 | 0.22 | 1.60 | 3.13 |
| White, Size #0 | | | | | | |
| #16 HPMC Capsule, White | 0.27 | 1.28 | 2.47 | 0.22 | 1.44 | 2.53 |
| #8, Size #0 | | | | | | |
| #17 Drug Substance | 0.35 | 1.38 | 2.72 | 0.36 | 1.67 | 3.39 |

Generally the dry mixtures had fewer stability issues than wet samples. Tests indicated that most of the processing excipients studied had limited effect on the chemical stability of the drug substance when used dry and are suitable for use in the development of solid-dose formulations.

### Example 8

### Direct Compression Tablets of Pimavanserin Tartrate Crystalline Form A

Formulations of 1 mg, 5 mg, and 20 mg of pimavanserin tartrate Form A suitable for direct compression into tablets were made using lactose, microcrystalline cellulose (AVICEL® PH102), and magnesium stearate as excipients. It was discovered that during blending, the mixture tended to form aggregates, potentially affecting content uniformity. Accordingly, pimavanserin tartrate Form A was blended with spray-dried lactose prior to sieving at 0.5 mm for 1 mg and 5 mg formulations and at 0.7 mm for 20 mg formulations. It was found that the stepwise blending/sieving procedure ensured that a homogenous blend was maintained. The blending times were set according to standard manufacturing procedures.

It was discovered that the flow properties could be improved by adding a silicon dioxide aerosol (AEROSIL®). Furthermore, it was discovered that increasing the magnesium stearate amount to 1.0 w/w % for the 1 mg formulation, 1.5 w/w % for the 5 mg formulation, and 2.0 w/w % for the 20 mg formulation improved blending by preventing seizing.

Tablets containing 1 mg, 5 mg, and 20 mg of pimavanserin tartrate Form A were made by first pre-blending the drug with spray-dried lactose as described above. The mixture was blended manually with microcrystalline cellulose (AVICEL® PH102) and silicon dioxide aerosol (AEROSIL®) and sieved with 1 mm net. The mixture was transferred to a double-cone blender and blended for 6 minutes. Magnesium stearate was charged to a stainless steel bow pre-blend with an equal volume of dry blend from the blender. The mixture was sieved manually with a 1 mm net. The sieved magnesium stearate blend was then added to the blender. Final blending was performed for 2 minutes. Direct compression of tablets was conducted using either a pilot scale rotary tablet press or a single punch lab scale machine. Table 5 indicates the final tablet compositions. The tablets were tested for content uniformity to monitor the efficiency of the blending/sieving procedures. The results of physical and analytical testing, depicted in Table 6, indicated acceptable tableting parameters.

TABLE 5
Final compositions (mg/tablet) of direct compression tablets.

| | Tablet strength | | |
|---|---|---|---|
| | 1 mg | 5 mg | 20 mg |
| Drug | 1.03 | 5.13 | 20.5 |
| Lactose monohydrate, | 87.5 | 82.9 | 66.5 |
| spray-dried | | | |
| Microcrystalline cellulose | 10.0 | 10.0 | 10.0 |
| Silicon dioxide aerosol | 0.50 | 0.50 | 1.00 |
| Magnesium stearate, | 1.00 | 1.50 | 2.00 |
| vegetable | | | |

TABLE 6
Physical and analytical characteristics of direct compression tablets.

| Tablet strength | 1 mg | 5 mg | 20 mg | 20 mg | 5 mg |
|---|---|---|---|---|---|
| LOD (%) | 1.4% | 1.7% | 1.8% | 1.8% | 1.8% |
| Bulk/Tapped density g/ml | 0.59/0.67 | 0.55/0.66 | 0.40/0.52 | 0.48/0.57 | 0.58/0.68 |
| Tablet weight | 99.9 mg | 99.8 mg | 101 mg | 101 mg | 101 mg |
| Crushing strength | 62N | 56N | 74N | 67N | 56N |
| Tablet height | 3.7 mm | 3.6 mm | 3.8 mm | 3.7 mm | 3.7 mm |
| Friability, 100 rot (%) | 0.1% A | 0% A | 0% A | — | — |
| Friability, 200 rot (%) | — | — | — | 0.1% A | 0% A |
| Disintegration time | 7 | 11 | 9 | 11 | 10 |
| Content uniformity, | 0.9 mg | 5.2 mg | 20.7 mg | — | 5.0 mg |
| mg/tablet | | | | | |
| Rsd (%) | 8.5% | 2.8% | 2.7% | — | 7.8% |
| Released amount %, 30 min | 93% | 106% | 108% | — | — |

LOD = limit of detection;
Rsd = relative standard deviation

### Example 9

### Wet Granulation Tablets of Pimavanserin Tartrate Crystalline Form A

Wet granulation formulations of 1 mg, 5 mg, and 20 mg of crystalline Form A were made for compression into tablets. Initial wet granulation formulations exhibited discoloration. Replacing lactose with mannitol did not remove the discoloration problem. It was discovered that replacing water as the granulation solvent with ethanol removed discoloration. Accordingly, it was determined that water was the source of the initial discoloration.

The drug product, pregelatinized starch (UNI-PURE® WG225), and mannitol (PEARLITOL® 200SD) were pre-blended in an intensive mixer. The mixture was granulated using a solution of povidone (KOLLIDON® 25) in ethanol (99.5%). The granulation was then dried in a drying cabinet at 40° C. The dried granulation was sieved with a 1.1 mm net and placed in a blender. Magnesium stearate was charged to a stainless steel bow pre-blend with and equal volume of dry blend form the blender. The resulting blend was sieved manually with a 1 mm net. The sieved magnesium stearate blend was added to the blender and final blending was performed for 2 minutes. The resulting mixture was then compressed into tablets. Table 7 indicates the ingredient amounts used in making the wet granulation formulations for 1 mg, 5 mg, and 20 mg tablets. The wet granulation formulations were tested for physical and analytical characteristics (Table 8). The results indicated acceptable analytical parameters.

TABLE 7
Ingredient amounts (%) for wet granulation formulations.

| | Tablet strength | | |
|---|---|---|---|
| | 1 mg | 5 mg | 20 mg |
| Drug | 1.03 | 5.13 | 20.5 |
| Mannitol | 91.3 | 87.2 | 71.1 |
| Pregelatinized Starch | 5.00 | 5.00 | 5.00 |
| Povidone | 1.15 | 1.15 | 1.15 |
| Magnesium stearate | 1.5 | 1.5 | 2.0 |
| Ethanol 99.5%** | 19.8 | 16.5 | 16 |

**Evaporates during manufacturing process

TABLE 8
Physical and analytical characteristics of wet granulation formulations.

| Tablet strength | 1 mg | 5 mg | 20 mg | 5 mg | 20 mg |
|---|---|---|---|---|---|
| LOD (%) | 1.5% | 0.8% | 2.3% | 0.9% | 1.6% |
| Sieving analysis (%) | | | | | |
| >710 | 0 | 0 | 0 | 0 | 0 |
| 500-710 | 0 | 0 | 1 | 0 | 1 |
| 250-500 | 8 | 5 | 7 | 4 | 6 |
| 125-250 | 69 | 69 | 69 | 71 | 72 |
| 90-125 | 19 | 21 | 18 | 21 | 18 |
| <90 | 5 | 4 | 6 | 5 | 4 |
| Bulk/Tapped density | 0.57/0.60 | 0.58/0.64 | 0.61/0.63 | 0.5610.60 | 0.56/0.61 |
| g/ml | | | | | |
| Tablet weight | 100 mg | 100 mg | 100 mg | 101 mg | 99.7 mg |
| Crushing strength | 61N | 51N | 58N | 61N | 44N |
| Tablet height | 3.7 mm | 3.8 mm | 3.8 mm | 3.7 mm | 3.8 mm |
| Friability, 100 rot. (%) | 0.1 | 0.1 | 0 | — | — |
| Friability, 200 rot. (%) | — | — | — | 0.1 | 0.1 |
| Disintegration time | 6 min | 6 min | 5 min | 6 min | 5 min |
| Content uniformity, | 1.0 mg | 5.4 mg | 20.5 mg | — | 20.1 mg |
| mg/tabl | | | | | |
| Released amount %, 30 min | 98% | 117% | 102% | — | — |

### Example 10

### Taste Masking Coating of Pimavanserin Tartrate Tablet Cores

A test panel was given small amounts of pimavanserin tartrate and it was noted that the compound has a pronounced bitter taste. Accordingly, a taste-masking coating was applied to direct-compressed 5 mg and 20 mg tablet cores. The coating system (OPADRY® White 06F28555) was water based and selected to minimize wetting of the tablet cores. The ingredients (including the coating formulation) of the resulting tablets are indicated in Table 9. Physical and analytical characteristics are indicated in Table 10.

TABLE 9
Ingredient amounts (mg/tablet) of coated direct compression tablets.

Tablet strength

| | 20 mg | 5 mg |
|---|---|---|
| Drug | 20.5 | 5.13 |
| Lactose monohydrate, | 66.5 | 82.9 |
| spray-dried | | |
| Microcrystalline cellulose | 10.0 | 10.0 |
| Silicon dioxide aerosol | 1.00 | 0.50 |
| Magnesium stearate, | 2.00 | 1.50 |
| vegetable | | |
| OPADRY ® White | 5 | 5 |
| 06F28555 | | |
| Water** | 50.6 | 50.6 |

**Evaporates during manufacturing process

TABLE 10
Physical and analytical characteristics of coated direct compression tablets.

| | | | |
|---|---|---|---|
| Tablet strength | 20 mg | 5 mg | 5 mg |
| Tablet weight | 105 mg | 106 mg | 105 mg |
| Film coating weight | 5 mg | 5 mg | 5 mg |
| Tablet height | 3.9 mm | 3.8 mm | 3.8 mm |
| Crushing strength | 118N | 115N | 102N |
| Disintegration time | 14 min | 16 min | 18 min |
| Content uniformity, | 20.0 mg | 5.0 mg | 5.0 mg |
| mg/tablet | | | |
| Rsd (%) | 6.7% | 7.9% | 3.9% |
| Released amount %, | 106% | — | 101% |
| 30 min | | | |

### Example 11

### Excipient Compatibility Study with Pimavanserin Tartrate Crystalline Form C

Binary physical mixtures of crystalline Form C and various excipients were prepared. Each binary blend were placed in amber glass vials and stored both uncapped and capped in stability chambers under controlled conditions (40° C.±2° C./75%±5% RH and 20° C.±2° C./60%±5% RH). The sampling time intervals were 0, 2, 4, and 8 weeks. Testing included assay, related substances, moisture content, and appearance. The results from the testing and compatibility conclusions are indicated in Table 11.

The assay test was conducted using a Waters XTerra RP₁₈ 250×4.6 mm, 5 μm column maintained at 30° C. The mobile phase was acetonitrile-5 mM ammonium acetate at pH 10.0. Pimavanserin was detected by UV-absorption at 226 nm. Quantification of pimavanserin was based on peak area comparison to an external standard solution of pimavanserin.

Related substances were determined using reversed-phase HPLC. The binary mixtures were dissolved in acetonitrile-water (15:85). Separation was achieved using gradient elution and a Waters Symmetry C₁₈, 150×4.6 mm, 3.5 μm column maintained at 35° C. Mobile phase A consisted of 1.00 ml trifluoracetic acid added to 1 L acetonitrile-water (15:85). Mobile phase B consisted of 1.00 ml trifluoroacetic acid added to 1 L acetonitrile. The gradient program included a linear ramp of 100% mobile phase A to 100% mobile phase B over 30 minutes, followed by 1 minute of isocratic flow, followed by a linear ramp of 100% mobile phase B to 100% mobile phase A in 2 minutes, followed by 17 minutes of isocratic flow. Pimavanserin and related substances were detected by UV-absorption at 226 nm. Related substances were calculated as a percentage (w/w) of the nominal pimavanserin content.

Moisture content was determined by a Karl Fischer method.

TABLE 11
Crystalline Form C excipient compatibility.

| | Time (weeks) | | | | |
|---|---|---|---|---|---|
| | | | | | Overall |
| Excipient | 0 | 2 | 4 | 8 | change |
| | Lactose monohydrate | | | | |
| Appearance | conforms | conforms | conforms | conforms | |
| Assay (% w/w) | 36.90 | 37.00 | 36.90 | 36.46 | −0.44 |
| Related | 0.16 | 0.11 | 0.20 | 0.40 | 0.24 |
| Substances (%) | | | | | |
| Moisture | 3.10 | 3.20 | 3.20 | 3.30 | 0.20 |
| content (%) | | | | | |
| Compatibility | Compatible | | | | |
| conclusion | | | | | |
| | Lactose anhydrous | | | | |
| Appearance | conforms | conforms | conforms | conforms | |
| Assay (% w/w) | 48.50 | 48.80 | 49.20 | 50.00 | 1.50 |
| Related | 0.19 | 0.17 | 0.44 | 0.39 | 0.20 |
| Substances (%) | | | | | |
| Moisture | 0.26 | 1.40 | 2.10 | 2.50 | 2.24 |
| content (%) | | | | | |
| Compatibility | Compatible | | | | |
| conclusion | | | | | |
| | AVICEL ® PH102 | | | | |
| Appearance | conforms | conforms | conforms | conforms | |
| Assay (% w/w) | 48.30 | 47.90 | 48.20 | 46.41 | −1.89 |
| Related | 0.22 | 0.21 | 0.29 | 0.43 | 0.21 |
| Substances (%) | | | | | |
| Moisture | 2.30 | 3.70 | 2.80 | 3.20 | 0.90 |
| content (%) | | | | | |
| Compatibility | Compatible | | | | |
| conclusion | | | | | |
| | AVICEL ® PH112 | | | | |
| Appearance | conforms | conforms | conforms | conforms | |
| Assay (% w/w) | 48.50 | 47.50 | 48.10 | 36.54 | −11.96 |
| Related | 0.23 | 0.17 | 0.30 | 0.49 | 0.26 |
| Substances (%) | | | | | |
| Moisture | 2.00 | 3.70 | 3.00 | 3.70 | 1.70 |
| content (%) | | | | | |
| Compatibility | Compatible | | | | |
| conclusion | | | | | |
| | CELLACTOSE ® 80 | | | | |
| Appearance | conforms | conforms | conforms | conforms | |
| Assay (% w/w) | 47.80 | 48.60 | 48.70 | 48.06 | 0.26 |
| Related | 0.20 | 0.19 | 0.28 | 0.79 | 0.59 |
| Substances (%) | | | | | |
| Moisture | 2.40 | 3.10 | 3.00 | 3.20 | 0.80 |
| content (%) | | | | | |
| Compatibility | Compatible | | | | |
| conclusion | | | | | |
| | STARCH 1500 ® | | | | |
| Appearance | conforms | conforms | conforms | conforms | |
| Assay (% w/w) | 49.50 | 50.20 | 50.00 | 44.34 | −5.16 |
| Related | 0.19 | 0.21 | 0.22 | 0.33 | 0.14 |
| Substances (%) | | | | | |
| Moisture | 3.90 | 5.90 | 5.20 | 5.80 | 1.90 |
| content (%) | | | | | |
| Compatibility | Compatible | | | | |
| conclusion | | | | | |
| | Calcium Phosphate, dibasic anhydrous | | | | |
| Appearance | conforms | conforms | conforms | conforms | |
| Assay (% w/w) | 49.00 | 48.00 | 47.80 | 48.41 | −0.59 |
| Related | 0.26 | 0.33 | 0.46 | 0.73 | 0.47 |
| Substances (%) | | | | | |
| Moisture | 0.06 | 0.13 | 0.29 | 0.17 | 0.11 |
| content (%) | | | | | |
| Compatibility | Compatible | | | | |
| conclusion | | | | | |
| | PROSOLV ® | | | | |
| Appearance | conforms | conforms | conforms | conforms | |
| Assay (% w/w) | 47.80 | 48.00 | 47.40 | 47.40 | −0.40 |
| Related | 0.34 | 0.24 | 0.39 | 0.71 | 0.37 |
| Substances (%) | | | | | |
| Moisture | 2.30 | 3.50 | 2.80 | 3.10 | 0.80 |
| content (%) | | | | | |
| Compatibility | Compatible | | | | |
| conclusion | | | | | |
| | Sodium Starch Glycolate | | | | |
| Appearance | conforms | does not | does not | does not | |
| | | conform | conform | conform | |
| Assay (% w/w) | 47.00 | 43.10 | 41.50 | 39.97 | −7.03 |
| Related | 0.14 | 2.72 | 4.75 | 8.03 | 7.89 |
| Substances (%) | | | | | |
| Moisture | 2.10 | 14.80 | 12.60 | 10.70 | 8.60 |
| content (%) | | | | | |
| Compatibility | Not compatible | | | | |
| conclusion | | | | | |
| | Croscarmellose sodium | | | | |
| Appearance | conforms | does not | does not | does not | |
| | | conform | conform | conform | |
| Assay (% w/w) | 46.70 | 43.20 | 44.00 | 42.64 | −4.06 |
| Related | 0.09 | 0.45 | 0.97 | 2.34 | 2.25 |
| Substances (%) | | | | | |
| Moisture | 1.30 | 9.90 | 9.90 | 10.20 | 8.90 |
| content (%) | | | | | |
| Compatibility | Not compatible | | | | |
| conclusion | | | | | |
| | Sodium lauryl sulfate | | | | |
| Appearance | conforms | does not | does not | does not | |
| | | conform | conform | conform | |
| Assay (% w/w) | 49.90 | 44.90 | 45.30 | 43.78 | −6.12 |
| Related | 0.12 | 0.23 | 0.34 | 0.59 | 0.47 |
| Substances (%) | | | | | |
| Moisture | 0.58 | 9.80 | 9.40 | 9.20 | 8.62 |
| content (%) | | | | | |
| Compatibility | Compatible | | | | |
| conclusion | | | | | |
| | Silicon dioxide | | | | |
| Appearance | conforms | conforms | conforms | conforms | |
| Assay (% w/w) | 50.90 | 52.00 | 48.60 | 42.20 | −8.70 |
| Related | 0.16 | 0.48 | 0.90 | 1.77 | 1.61 |
| Substances (%) | | | | | |
| Moisture | 0.43 | 0.89 | 0.72 | 0.74 | 0.31 |
| content (%) | | | | | |
| Compatibility | Not compatible | | | | |
| conclusion | | | | | |
| | Magnesium Stearate | | | | |
| Appearance | conforms | conforms | conforms | does not | |
| | | | | conform | |
| Assay (% w/w) | 50.60 | 47.90 | 48.30 | 48.24 | −2.36 |
| Related | 0.27 | 0.78 | 1.16 | 1.66 | 1.39 |
| Substances (%) | | | | | |
| Moisture | 2.60 | 4.80 | 4.90 | 4.50 | 1.90 |
| content (%) | | | | | |
| Compatibility | Compatible | | | | |
| conclusion | | | | | |
| | Sodium stearyl fumarate | | | | |
| Appearance | conforms | conforms | conforms | does not | |
| | | | | conform | |
| Assay (% w/w) | 50.80 | 50.20 | 50.30 | 49.77 | −1.03 |
| Related | 0.47 | 0.15 | 0.21 | 0.55 | 0.41 |
| Substances (%) | | | | | |
| Moisture | 1.50 | 1.50 | 1.60 | 1.60 | 0.10 |
| content (%) | | | | | |
| Compatibility | Compatible | | | | |
| conclusion | | | | | |
| | Talc | | | | |
| Appearance | conforms | conforms | conforms | conforms | |
| Assay (% w/w) | 50.30 | 49.70 | 49.30 | 49.80 | −0.50 |
| Related | 0.18 | 0.22 | 0.25 | 0.40 | 0.22 |
| Substances (%) | | | | | |
| Moisture | 0.15 | 0.17 | 0.16 | 0.17 | 0.02 |
| content (%) | | | | | |
| Compatibility | Compatible | | | | |
| conclusion | | | | | |
| | Opadry | | | | |
| Appearance | conforms | conforms | conforms | conforms | |
| Assay (% w/w) | 47.30 | 47.20 | 47.20 | 47.95 | 0.65 |
| Related | 0.36 | 0.31 | 0.60 | 0.73 | 0.37 |
| Substances (%) | | | | | |
| Moisture | 0.91 | 2.10 | 1.40 | 2.10 | 1.19 |
| content (%) | | | | | |

| | |
|---|---|
| Compatibility | Compatible |
| conclusion | |

### Example 12

### Direct Compression Tablets of Crystalline Form C of Pimavanserin Tartrate

Several different formulations containing crystalline Form C of pimavanserin tartrate are prepared using dry blending of excipients and direct compression into tablets. The ingredients include one or more of STARCH 1500®, lactose, PROSOLV® 90, PROSOLV® HD90, or PROSOLV® 50. The tablets are coated with a taste masking film.

The ingredient amounts for four formulations containing lactose, PROSOLV® (90 or HD90), and magnesium stearate are listed in Tables 15-19.

TABLE 15
1 mg strength tablet of -(1-methylpiperidin-4-yl)-N-(4-
fluorophenylmethyl)-N′-(4- (2-methylpropyloxy)
phenylmethyl) carbamide tartrate Form C.

| Ingredients | mg/tablet | % w/w |
|---|---|---|
| Drug | 1 | 1 |
| Lactose monohydrate | 88 | 88 |
| PROSOLV ® 90 | 10.0 | 10.0 |
| Magnesium stearate | 1.00 | 1.00 |

TABLE 16
1 mg strength tablet of pimavanserin tartrate Form C.

| Ingredients | mg/tablet | % w/w |
|---|---|---|
| Drug | 1 | 0.67 |
| Lactose monohydrate | 138 | 92.00 |
| PROSOLV ® HD90 | 10.0 | 6.67 |
| Magnesium stearate | 1.00 | 0.67 |

TABLE 17
20 mg strength tablet of pimavanserin tartrate Form C.

| Ingredients | mg/tablet | % w/w |
|---|---|---|
| Drug | 20 | 13.33 |
| Lactose monohydrate | 113 | 75.33 |
| PROSOLV ® HD90 | 15.0 | 10.00 |
| Magnesium stearate | 2.00 | 1.33 |

TABLE 18
1 mg strength tablet of pimavanserin tartrate Form C.

| Ingredients | % w/w |
|---|---|
| Drug | 1 |
| Lactose monohydrate | 90.5 |
| PROSOLV ® 90 | 8 |
| Magnesium stearate | 0.5 |

TABLE 19
20 mg strength tablet of pimavanserin tartrate Form C.

| Ingredients | % w/w |
|---|---|
| Drug | 20 |
| Lactose monohydrate | 69 |
| PROSOLV ® 90 | 10 |
| Magnesium stearate | 1 |

Tablets containing PROSOLV® (50 or HD90), STARCH 1500®, and magnesium stearate are made having the amounts listed in Tables 20-24. STARCH 1500®, Crystalline Form C, and PROSOLV® HD90 (portion I) are placed, in the order listed, into a V-blender and the mixture blended for 5 min. The blend (Dry Blend I) is discharged into a suitable polyethylene-lined container and the weight recorded. Dry Blend I is then passed through either a Sweco sifter fitted with a 30 mesh screen or through a 30 mesh hand screen into a suitable polyethylene-lined container. PROSOLV® HD90 (portion II), Dry Blend I, and PROSOLV® HD90 (portion III) are placed, in the order listed, into a V-blender, and the mixture blended for 10 min, before being discharged into a suitable polyethylene-lined container and the weight recorded. The blend (Dry Blend II) is then passed through a Sweco fitted with a 30 mesh screen into a suitable polyethylene-lined container. PROSOLV® HD90 (portion IV), Dry Blend II, and PROSOLV® HD90 (portion V) are placed, in the order listed, into a V-blender, and the mixture blended for 10 min, before being discharged into a suitable polyethylene-lined container and the weight recorded. The blend (Dry Blend III) is then passed through a Sweco fitted with a 30 mesh screen into a suitable polyethylene-lined container. The blend (Dry Blend III) is placed into a V-blender and blended for 10 min. Magnesium stearate is passed through a 40 mesh hand screen and added to the V-blender and the mixture blended for 5 min, before the powder is discharged into a suitable polyethylene-lined container and the weight recorded. The blended powders are compressed on a Manesty EXPRESS25 ( 9/32 inch round tooling) at a press speed of 30 rpm for the 5 mg and 20 mg tablets and at a speed of 30-60 rpm for the 1 mg tablets.

A coating suspension is prepared by adding Opadry slowly to purified water, with agitation, followed by mixing of the suspension for at least 45 min. The batch of tablet cores is divided into two equal sized sub-batches. Coating is performed in a Compu-Lab coating pan using the following settings for the coating parameters: Inlet air temperature: 60° C. (range: 50-80° C.); Air flow rate: 250 cfm (range: 150-400 cfm); Pan speed: 10 rpm (range: 8-20 rpm); Spray rate: 40-140 g/min; Exhaust temperature: 45° C. (range: 36-48° C.); Bed temperature: 40° C. (range: 36-48° C.); and Air atomization: 25 psi (range: 15-30 psi). The two sub-batches of coated tablets are combined into one batch of final coated tablets.

TABLE 20
1 mg strength tablet of pimavanserin tartrate Form C.

| Ingredients | mg/tablet | % w/w |
|---|---|---|
| Drug | 1 | 1 |
| PROSOLV ® 50 | 90.5 | 90.5 |
| Starch 1500 | 8.0 | 8.0 |
| Magnesium stearate | 0.5 | 0.5 |

TABLE 21
1 mg strength tablet of pimavanserin tartrate Form C.

| Ingredients | mg/tablet | % w/w |
|---|---|---|
| Drug | 1 | 0.67 |
| PROSOLV ® HD90 | 140.5 | 93.67 |
| Starch 1500 | 8.0 | 5.33 |
| Magnesium stearate | 0.5 | 0.33 |

TABLE 22
20 mg strength tablet of pimavanserin tartrate Form C.

| Ingredients | mg/tablet | % w/w |
|---|---|---|
| Drug | 20 | 13.33 |
| PROSOLV ® HD90 | 120 | 80.00 |
| Starch 1500 | 8.0 | 5.33 |
| Magnesium stearate | 2.0 | 1.33 |

TABLE 23
5 mg strength tablet of pimavanserin tartrate Form C.

| Ingredients | mg/tablet | % w/w |
|---|---|---|
| Drug | 5 | 3.33 |
| PROSOLV ® HD90 | 136 | 90.67 |
| Starch 1500 | 8.0 | 533 |
| Magnesium stearate | 1.0 | 0.67 |

TABLE 24
1 mg strength tablet of pimavanserin tartrate Form C.

| Ingredients | mg/tablet | % w/w |
|---|---|---|
| Drug | 1 | 0.67 |
| PROSOLV ® HD90 | 140 | 93.33 |
| Starch 1500 | 8.0 | 5.33 |
| Magnesium stearate | 1.0 | 0.67 |

### Example 13

### Dissolution Tests

A 150 mg tablet containing polymorph C was manufactured and subjected to a dissolution test. The results indicate that greater than 90% release of polymorph C occurred within 20 minutes.

### Example 14

### Impurity Measurements

100 mg tablets containing polymorphs A or C and packaged in blister packages were exposed to 75% relative humidity at 40° C. In tablets containing polymorph A, impurity 1 increased from 0.3% at the start of the experiment to 0.8% at 1 month and to 1.3% at 3 months. In contrast, in tablets containing polymorph C, impurity 1 increased from 0.14% at the start of the experiment to 0.17% after 1 month.

In another experiment, 100 mg tablets containing polymorph A were exposed to 60% relative humidity at 25° C. The amount of impurity 2 increased from 0.1% to 0.3% over time. In contrast, when 100 mg tablets containing polymorph C were exposed to 65% relative humidity at 30° C., impurity 2 could not be detected even after 11 weeks.

The structures of impurities 1 and 2 are as follows:

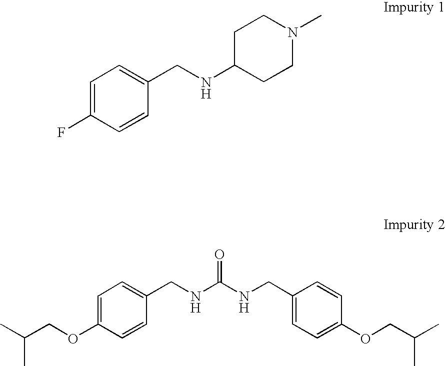

What is claimed is:

1. A pharmaceutical composition, comprising:
pimavanserin; and
at least one pharmaceutically acceptable excipient selected from the group consisting of a sugar, a starch, a cellulose preparation, silicon dioxide aerosol, gelatin, calcium phosphate dibasic, sodium lauryl sulfate, magnesium stearate, sodium stearyl fumarate, talc, polyethylene glycol, and polyvinylpyrrolidone, and combinations thereof.

2. The composition of claim 1, wherein the pharmaceutically acceptable excipient is selected from the group consisting of a sugar, pregelatinized starch, partially pregelatinized starch, microcrystalline cellulose, silicified microcrystalline cellulose, a lactose-cellulose blend, methyl cellulose, silicon dioxide aerosol, gelatin, calcium phosphate dibasic, sodium lauryl sulfate, magnesium stearate, sodium stearyl fumarate, talc, polyethylene glycol, and polyvinylpyrrolidone, and combinations thereof.

3. The composition of claim 2, wherein the silicified microcrystalline cellulose is PROSOLV® 90 or PROSOLV® 50.

4. The composition of claim 2, wherein the silicified microcrystalline cellulose is PROSOLV® HD90.

5. The composition of claim 2, wherein the silicified microcrystalline cellulose comprises microcrystalline cellulose, colloidal silicon dioxide, colloidal anhydrous silica, and light anhydrous silicic acid.

6. The composition of claim 2, wherein the partially pregelatinized starch is STARCH 1500®.

7. The composition of claim 2, wherein the lactose-cellulose blend is CELLACTOSE® 80.

8. The composition of claim 1, wherein the pharmaceutically acceptable excipient is selected from the group consisting of pregelatinized starch, partially pregelatinized starch, silicified microcrystalline cellulose, a lactose-cellulose blend, methyl cellulose, sodium stearyl fumarate, and polyvinylpyrrolidone, and combinations thereof.

9. The pharmaceutical composition of claim 1, wherein the pimavanserin is pimavanserin tartrate.

10. The pharmaceutical composition of claim 9, wherein the pimavanserin tartrate is crystalline Form A.

11. The pharmaceutical composition of claim 9, wherein the pimavanserin tartrate is crystalline Form C.

12. The pharmaceutical composition of claim 1, comprising a silicon dioxide aerosol.

13. The composition of claim 12, comprising at least about 0.1% by weight of the silicon dioxide aerosol.

14. The composition of claim 12, comprising at least about 0.5% by weight of the silicon dioxide aerosol.

15. The composition of claim 12, comprising at least about 1.0% by weight of the silicon dioxide aerosol.

16. The composition of claim 12, wherein the silicon dioxide aerosol has a specific surface area from about 175 to about 225 m²/g.

17. The composition of claim 16, wherein the silicon dioxide aerosol is AEROSIL® 200.

18. The composition of claim 12, comprising lactose, microcrystalline cellulose, and magnesium stearate.

19. The composition of claim 18, comprising at least about 50% by weight lactose, at least about 5% by weight microcrystalline cellulose, and at least about 0.5% by weight magnesium stearate.

20. The composition of claim 18, comprising at least about 65% by weight lactose, at least about 10% by weight microcrystalline cellulose, and at least about 1% by weight magnesium stearate.

21. The composition of claim 1, comprising lactose, magnesium stearate, and silicified microcrystalline cellulose.

22. The composition of claim 21, comprising at least about 50% by weight lactose, at least about 0.5% by weight magnesium stearate, and at least about 5% by weight silicified microcrystalline cellulose.

23. The composition of claim 21, comprising at least about 65% by weight lactose, at least about 1% by weight magnesium stearate, and at least about 10% by weight silicified microcrystalline cellulose.

24. The composition of claim 1, comprising partially pregelatinized starch, magnesium stearate, and silicified microcrystalline cellulose.

25. The composition of claim 24, comprising at least about 50% by weight silicified microcrystalline cellulose, at least about 0.5% by weight magnesium stearate, and at least about 5% by weight partially pregelatinized starch.

26. The composition of claim 24, comprising at least about 65% by weight silicified microcrystalline cellulose, at least about 0.5% by weight magnesium stearate, and at least about 5% by weight partially pregelatinized starch.

27. The composition of claim 24, comprising at least about 75% by weight silicified microcrystalline cellulose, at least about 1.0% by weight magnesium stearate, and at least about 5% by weight partially pregelatinized starch.

28. The composition of claim 1, comprising an additional antipsychotic agent.

29. The composition of claim 28, wherein the additional antipsychotic agent is selected from the group consisting of chlorpromazine, mesoridazine, prochlorperazine, thioridazine, Fluphenazine, Perpehnazine, Trifluoperazine, haloperidol, pimozide, clozapine, loxapine, olanzapine, quetiapine, resperidone, ziprasidone, lithium carbonate, Aripiprazole, ETRAFON®, Droperidol, Thioridazine, Thiothixene, Promethazine, Metoclopramide, Chlorprothixene, TRIAVIL®, Molindone, Sertindole, Droperidol, Amisulpride, Melperone, Paliperidone, and Tetrabenazine.

30. A method of treating or preventing a condition selected from the group consisting of a neuropsychiatric disorder, a neurodegenerative disorder, and an extrapyramidal disorder, comprising administering to a subject the pharmaceutical composition of claim 1.

31. A method of reducing a side effect of an antipsychotic agent, comprising administering to a subject the pharmaceutical composition of claim 1.

32. The method of claim 31, wherein the side effect is selected from the group consisting of stroke, tremors, sedation, gastrointestinal problems, neurological problems, increased risk of death, cerebrovascular events, movement disorder, dystonia, akathisia, parkinsoniam movement disorder, tardive dyskinesia, cognitive disorders, prolactinemia, catalepsy, psychosis, neuroleptic malignant syndrome, heart problems, pulmonary problems, diabetes, liver failure, suicidality, sedation, orthostatic hypotension, choking, dizziness, tachycardia, blood abnormalities (including abnormal triglyceride levels, increased cholesterol levels, dyslipidemia, and hyperglycemia), syncope, seizures, dysphagia, priapism, thrombotic thrombocytopenic purpura, disruption of body temperature regulation, insomnia, agitation, anxiety, somnolence, aggressive reaction, headache, constipation, nausea, dyspepsia, vomiting, abdominal pain, saliva increase, toothache, rhinitis, coughing, sinusitis, pharyngitis, dyspnea, back pain, chest pain, fever, rash, dry skin, seborrhea, increased upper respiratory infection, abnormal vision, arthralgia, hypoaesthesia, manic reaction, concentration impairment, dry mouth, pain, fatigue, acne, pruritus, myalgia, skeletal pain, hypertension, diarrhea, confusion, asthenia, urinary incontinence, sleepiness, increased duration of sleep, accommodation disturbance, palpitations, erectile dysfunction, ejaculatory dysfunction, orgastic dysfunction, lassitude, increased pigmentation, increased appetite, automatism, increased dream activity, diminished sexual desire, nervousness, depression, apathy, catatonic reaction, euphoria, increased libido, amnesia, emotional liability, nightmares, delirium, yawning, dysarthria, vertigo, stupor, paraesthesia, aphasia, hypoesthesia, tongue paralysis, leg cramps, torticollis, hypotonia, coma, migrain, hyperreflexia, choreoathetosis, anorexia, flatulence, stomatitis, melena, hemorrhoids, gastritis, fecal incontinence, erutation, gastroeophageal reflux, gastroenteritis, esophagitis, tongue discoloration, choleithiasis, tongue edema, diverticulitis, gingivitis, discolored feces, gastrointestinal hemorrhage, hematemesis, edema, rigors, malaise, pallor, enlarged abdomen, ascites, sarcoidosis, flushing, hyperventilation, bronchospasm, pneumonia, tridor, asthma, increased sputum, aspiration, photosensitivity, increased sweating, acne, descreased sweating, alopecia, hyperkeratosis, skin exfoliation, bullous eruption, skin ulceration, aggravated psoriasis, furunculosis, verruca, dermatitis lichenoid, hypertrichosis, genital pruritus, urticaria, ventricular tachycardia, angina pectoris, premature atrial contractions, T wave inversion, ventricular extrasystoles, ST depression, AV block, myocarditis, abnormal accommodation, xerophthalmia, diplopia, eye pain, blepharitis, photopsia, photophobia, abnormal lacrimation, hyponatremia, creatine phosphokinase increase, thirst, weight decrease, decreased serum iron, cachexia, dehydration, hypokalemia, hypoproteinemia, hyperphosphatemia, hypertrigylceridemia, hyperuricemia, hypoglycemia, polyuria, polydipsia, hemturia, dysuria, urinary retention, cystitis, renal insufficiency, arthrosis, synostosis, bursitis, arthritis, menorrhagia, dry vagina, nonpeurperal lactation, amenorrhea, female breast pain, leukorrhea, mastitis, dysmenorrhea, female perineal pain, intermenstrual bleeding, vaginal hemorrhage, increased SGOT, increased SGPT, cholestatic hepatitis, cholecystitis, choleithiasis, hepatitis, hepatocellular damage, epistaxis, superficial phlebitis, thromboplebitis, thrombocytopenia, tinnitus, hyperacusis, decreased hearing, anemia, hypochromic anemia, normocytic anemia, granulocytopenia, leukocytosis, lymphadenopathy, leucopenia, Pelger-Huet anomaly, gynecomastia, male breast pain, antiduretic hormone disorder, bitter taste, micturition disturbances, oculogyric crisis, abnormal gait, involuntary muscle contraction, and increased injury.

33. A pharmaceutical composition, comprising:
pimavanserin tartrate; and
at least about 0.5% of a lubricant.

34. The composition of claim 33, comprising at least about 0.8% by weight of the lubricant.

35. The composition of claim 33, comprising at least about 1% by weight of the lubricant.

36. The composition of claim 33, comprising at least about 1.5% by weight of the lubricant.

37. The composition of claim 33, comprising at least about 2% by weight of the lubricant.

38. The composition of claim 33, wherein the lubricant is magnesium stearate.

39. The composition of claim 33, wherein the lubricant is sodium stearyl fumarate.

40. A wet granulation formulation for use in preparing tablets, the formulation comprising:
pimavanserin tartrate; and
a non-aqueous granulation solvent.

41. The formulation of claim 40, wherein the non-aqueous granulation solvent comprises ethanol.

42. The formulation of claim 40, further comprising mannitol or lactose, pregelatinized or partially pregelatinized starch, and povidone.

43. The formulation of claim 40, further comprising at least about 65% by dry weight mannitol, at least about 2% by dry weight pregelatinized or partially pregelatinized starch, and at least about 0.5% by dry weight povidone.

44. The formulation of claim 40, further comprising at least about 70% by dry weight mannitol, at least about 5% by dry weight pregelatinized or partially pregelatinized starch, and at least about 1% by dry weight povidone.

45. A wet granulation formulation for use in preparing tablets, the formulation comprising:
pimavanserin tartrate; and
less then about 30% by weight of water.

46. The formulation of claim 45, comprising substantially no water.

47. A method of preparing a pharmaceutical tablet, the method comprising:
granulating pimavanserin tartrate using a non-aqueous granulation solvent;
drying the granulation;
blending the granulation with a lubricant; and
compressing the blend into a tablet.

48. The method of claim 47, wherein the non-aqueous granulation solvent comprises ethanol.

49. The method of claim 47, wherein povidone is dissolved in the non-aqueous granulation solvent.

50. The method of claim 47, wherein the granulation further comprises mannitol or lactose, pregelatinized starch, and povidone.

51. The method of claim 47, wherein the lubricant comprises magnesium stearate.

52. A method of preparing a pharmaceutical tablet, the method comprising:
granulating pimavanserin tartrate using less than about 30% by weight of water;
drying the granulation;
blending the granulation with a lubricant; and
compressing the blend into a tablet.

53. A method of preparing a pharmaceutical tablet, the method comprising:
dry blending pimavanserin tartrate with at least one pharmaceutically acceptable excipient selected from the group consisting of a sugar, microcrystalline cellulose, lactose-cellulose blend, calcium phosphate dibasic, silicified microcrystalline cellulose, pregelatinized starch, partially pregelatinized starch, polyvinylpyrrolidone, HPMC, sodium lauryl sulfate, sodium stearyl fumerate, silicon dioxide aerosol, magnesium stearate, talc, polyethylene glycol, and combinations thereof; and
compressing the blend to form a tablet.

54. The method of claim 53, further comprising coating the tablet with a taste-masking film.

55. A pharmaceutical composition, comprising:
pimavanserin; and
a pharmaceutically acceptable excipient, wherein the pharmaceutical composition comprises substantially no sodium starch glycolate or sodium croscarmellose.

56. The composition of claim 55, wherein the pimavanserin is pimavanserin tartrate.

57. A pharmaceutical tablet, comprising:
a core comprising pimavanserin; and
a taste-masking film coating over the core.

58. The tablet of claim 57, where the film coating is an OPADRY® film.

59. The tablet of claim 57, wherein the pimavanserin is pimavanserin tartrate.

60. A pharmaceutical composition, comprising:
pimavanserin or a pharmaceutically acceptable salt thereof; and
a pharmaceutically acceptable excipient;
wherein the pharmaceutical composition comprises less than about 0.1% of a compound having the structure of Impurity 2:

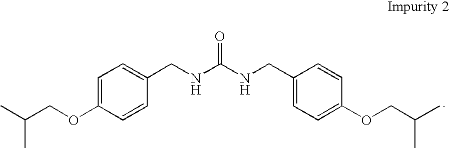

61. The composition of claim 60, wherein the pharmaceutical composition is substantially free of Impurity 2.

62. The composition of claim 60, wherein the pharmaceutical composition is substantially free of Impurity 2 after storage in a blister package at about 30° C. and about 65% relative humidity for at least about 10 weeks.

63. A pharmaceutical composition, comprising:
pimavanserin or a pharmaceutically acceptable salt thereof; and
a pharmaceutically acceptable excipient;
wherein the pharmaceutical composition comprises less than about 0.25% of a compound having the structure of Impurity 1:

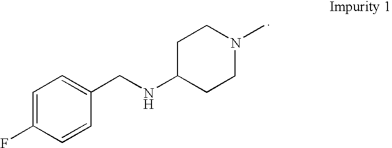

64. The composition of claim 63, wherein the pharmaceutical composition comprises less than about 0.25% of Impurity 1 after storage in a blister package at about 40° C. and about 75% relative humidity for at least about 1 month.

65. A pharmaceutical composition, comprising pimavanserin and at least one pharmaceutically acceptable excipient, wherein the composition is formulated such that at least about 80% of the pimavanserin is released from the composition upon administration to a subject.

66. The composition of claim 65, wherein the composition is formulated such that at least about 90% of the pimavanserin is released from the composition upon administration to a subject.
